(19) United States
(12) Patent Application Publication
(10) Pub. No.: US 2008/0138415 A1
(43) Pub. Date: Jun. 12, 2008

(54) COMPOSITION AND METHOD FOR PRODUCING COMPOSITION FOR CONSTRUCTING TISSUE, AND TISSUE CONSTRUCT

(76) Inventors: Mehboob HUSSAIN, Baltimore, MD (US); Robert G. DENNIS, Raleigh, NC (US); David A. WOLF, Houston, TX (US)

Correspondence Address: LADAS & PARRY LLP, 224 SOUTH MICHIGAN AVENUE, SUITE 1600, CHICAGO, IL, 60604, US

(21) Appl. No.: 11/859,952

(22) Filed: Sep. 24, 2007

Related U.S. Application Data
(60) Provisional application No. 60/847,250, filed on Sep. 26, 2006.

(51) Int. Cl. A61K 35/12; A61K 9/14; C12N 5/06; C12N 13/00; A61P 43/00

(52) U.S. Cl. 424/486; 424/93.7; 435/377; 435/366; 435/173.1

(57) ABSTRACT

A cellular composition comprising a mesenchymal stem cell product and a biogel with methods for preparing and for using the composition. Tissue constructs made with the composition are also disclosed.

### FIELD OF THE INVENTION

The present invention relates to a cellular composition comprising a mesenchymal stem cell product and a biogel, methods of preparing a mesenchymal stem cell product and the products made by those methods. The present invention also relates to a method for the administration of a cellular composition to a mammal and/or the use of the composition for the preparation of a medicament for the construction of mammalian tissue. A composition for constructing tissue is also included in the present invention.

### BACKGROUND OF THE INVENTION

Cell culture processes have been developed for the increase in number of single cell bacteria, yeast and molds which are resistant to environmental stresses or are encased with a tough cell wall. Mammalian cell culture, however, is much more complex because such cells are more delicate and have more complex nutrient and other environmental requirements in order to maintain viability and cell growth. Large-scale cultures of bacterial type cells are highly developed and such culture processes are less demanding and are not as difficult to cultivate as mammalian cells. These techniques are highly empirical and a firm theoretical basis is not developed. Bacterial cells can be grown in large volumes of liquid medium and can be vigorously agitated without any significant damage. Mammalian cells, on the other hand, cannot withstand excessive turbulent action without damage to the cells and must be provided with a complex nutrient medium in culture.

In addition, mammalian cells have other special requirements; in particular most mammal cells must attach themselves to some substrate surface to remain viable and to duplicate. On a small scale, mammalian cells have been grown in containers with small microwells to provide surface anchors for the cells. However, cell culture processes for mammalian cells in such microwell containers generally do not provide sufficient surface area to grow mammalian cells on a sufficiently large scale basis for many commercial or research applications. To provide greater surface areas, microcarrier beads have been developed for providing increased surface areas for the cultured cells to attach. Microcarrier beads with attached cultured cells require agitation in a conventional bioreactor vessel to provide suspension of the cells, distribution of fresh nutrients, and removal of metabolic waste products. To obtain agitation, such bioreactor vessels have used internal propellers or movable mechanical agitation devices which are motor driven so that the moving parts within a vessel cause agitation in the fluid medium for the suspension of the microcarrier beads and attached cells. Agitation of mammalian cells, however, subjects them to high degrees of shear stress that can damage the cells and limit ordered assembly of these cells according to cell derived energy. These shear stresses arise when the fluid media has significant relative motion with respect to vessel walls, impellers, or other vessel components. Cells may also be damaged in bioreactor vessels with internal moving parts if the cells or beads with cells attached collide with one another or vessel components.

In addition to the drawbacks of cell damage, bioreactors and other methods of culturing mammalian cells are also very limited in their ability to provide conditions that allow cells to assemble into tissues that simulate the spatial 3-dimensional form of actual tissues in the intact organism. Conventional tissue culture processes limit, for similar reasons, the capacity for cultured tissues to express a highly functionally specialized or differentiated state considered that may be important for mammalian cell differentiation and secretion of specialized biologically active molecules of research and pharmaceutical interest. Unlike microorganisms, the cells of higher organisms such as mammals form themselves into high order multicellular tissues. Although the exact mechanisms of this self-assembly are not known, in the cases that have been studied thus far, development of cells into tissues has been found to be dependent on orientation of the cells with respect to each other (the same or different type of cell) or other anchorage substrate and/or the presence or absence of certain substances (factors) including but not limited to hormones, autocrines or paracrines (where for instance autocrines and/or paracrines includes for instance growth factors, cytokines, neutrophins). In summary, conventional culture processes do not achieve sufficiently low shear stress, sufficient freedom for 3-dimensional spatial orientation, freedom for localization of cells with differing sedimentation properties, and sufficiently long periods for critical cell interactions (with each other or substrates) to allow excellent modeling of in vivo tissue structure.

Mesenchymal stem cells or marrow stromal cells, are stem cells that can differentiate into osteoblasts, chondrocytes, myocytes, adipocytes, neuronal cells, and, as described lately, into beta-pancreatic islets cells. Mesenchymal stem cells differentiate from colony-forming unit-fibroblast (CFU-F). Mesenchymal stem cells cultured in the presence of transformation growth factor (TGF), preferably bone morphogenetic protein (BMP), will differentiate into chondrocytes, whereas mesenchymal stem cells cultured in serum with ascorbic acid, inorganic phosphate and dexamethasone will differentiate into osteoblasts. Mesenchymal stem cells have the capability for renewal and differentiation into various lineages of mesenchymal tissues. These features of mesenchymal stem cells attract a lot of attention from investigators in the context of cell-based therapies of several human diseases. Bone marrow represents the main available source of mesenchymal stem cells, although the use of bone-marrow-derived cells is not always acceptable due to the high degree of viral infection and the significant drop in cell number and proliferative/differentiation capacity with age.

### SUMMARY OF THE INVENTION

The present invention relates to a composition for reconstructing human tissue. More specifically, this invention relates to a composition for reconstructing human tissue, with the composition comprising a mixture of expanded mesenchymal stem cells and a biogel. A growth promoting agent can optionally be added to the mixture.

The present invention further relates to methods of expanding mesenchymal stem cells in a culture environment, either with or without a gravity field, with minimum fluid shear stress, freedom for three-dimensional spatial orientation of the suspended mesenchymal stem cells and localization of mesenchymal stem cells with differing or similar sedimentation properties in a similar spatial region. In one embodiment, a time varying electromagnetic field is applied to the expanding mesenchymal stem cells.

The present invention relates to a process for expanding mesenchymal stem cells within a culture medium, mixing the expanded mesenchymal stem cells with a biogel and constructing tissue with the mixture. The mesenchymal stem cells are preferably exposed to a time-varying electromagnetic field.

The present invention further relates to a process for the expansion of mesenchymal stem cells that are cultured, increased in number, and/or differentiated.

Other aspects, features and advantages of the present invention will be apparent from the following description of the embodiments of the invention given for the purpose of disclosure.

### BRIEF DESCRIPTION OF THE DRAWINGS

In the drawings,

FIG. 1 is a cross-sectional elevated side view of a preferred embodiment of a rotatable bioreactor;

FIG. 2 is an elevated side perspective of a preferred embodiment of a rotatable bioreactor;

FIG. 3 schematically illustrates a preferred embodiment of a culture medium flow loop;

FIG. 4 is a vertical cross-sectional view of a preferred embodiment of a rotatable bioreactor;

FIG. 5 is a cross section along line 3-3 of FIG. 4;

FIG. 6 is a vertical cross sectional view of a preferred embodiment of a rotatable bioreactor;

FIG. 7 is a vertical cross sectional view of a rotatable bioreactor;

FIG. 8 is a side view of a TVEMF-device;

FIG. 9 is an elevated front view of the TVEMF-device shown in FIG. 8;

FIG. 10 is an elevated front view of the TVEMF-device also shown in FIGS. 8 and 9 further showing a rotatable bioreactor adjacently located therein;

FIG. 11 is the orbital path of a typical cell in a non-rotating reference frame;

FIG. 12 is a graph of the magnitude of deviation of a cell per revolution; and

FIG. 13 is a representative cell path as observed in a rotating reference frame of the culture medium.

### DETAILED DESCRIPTION OF THE DRAWINGS

FIGS. 1 to 18 disclose preferred embodiments of a bioreactor of the present invention. A preferred bioreactor of the present invention is a rotatable bioreactor comprising a culture chamber, preferably substantially cylindrical, having at least one aperture, an interior portion, and an exterior portion wherein the interior portion defines a space that removably receives a cell mixture. The rotatable bioreactor has a motor that, in use, rotates the culture chamber about a horizontal axis, preferably horizontal longitudinal to create, foster, and maintain a three-dimensional culture therein. In a preferred embodiment, the culture chamber is encompassed by an electrically conductive coil, and the rotatable bioreactor has a time varying electromagnetic force (TVEMF) source operatively connected to the electrically conductive coil. The TVEMF source is operatively connected to the electrically conductive coil so that, in use, the TVEMF source delivers a TVEMF to the interior portion of the culture chamber and to the three-dimensional culture to expand and/or differentiate the cells therein.

In the drawings, referring now to FIG. 1, FIG. 1 is a cross sectional elevated side view of a preferred embodiment of a rotatable bioreactor 10. In this preferred embodiment a motor housing 12 is supported by a base 14. A motor 16 is attached inside the motor housing 12 and connected by a first wire 18 and a second wire 20 to a control box 22 that houses a control device therein whereby the speed of the motor 16 can be incrementally controlled by turning the control knob 24. Extending from the motor housing 12 is a motor shaft 26. A rotatable mounting 28 removably receives a rotatable bioreactor holder 30 that removably receives a culture chamber 32 preferably disposable and cylindrical, which is affixed, preferably removably, within the rotatable bioreactor holder 30, preferably by a screw 34. The culture chamber 32 is mounted, preferably removably, to the rotatable mounting 28. The rotatable mounting 28 is received by the motor shaft 26. When the control knob 24 is turned on, the culture chamber 32 is rotated about a horizontal axis at a speed that preferably fosters, supports, and maintains the cells three-dimensional geometry and cell to cell support and geometry while at the same time preventing cell collision with the interior portion of the rotatable bioreactor and with other cells. The horizontal axis is parallel to the plane of the earth.

The culture chamber of a rotatable bioreactor of the present invention may preferably be disposable wherein it can be discarded and a new one used in later cell cultures. The rotatable bioreactor (as a whole or in part) may also preferably be sterilized, for instance in an autoclave, after each use and re-used for later cell cultures. A disposable culture chamber could be manufactured and packaged in a sterile environment thereby enabling it to be used by a medical or research professional much the same as other disposable medical devices are used.

FIG. 1 also illustrates a TVEMF source which in this embodiment comprises an electrically conductive coil 35 wrapped around the exterior portion of the culture chamber 32. Preferably, an electrically conductive coil that is integral with a disposable culture chamber is installed into the bioreactor along with the disposable culture chamber and operatively connected to the controlling circuit 44. When the disposable culture chamber is discarded, the electrically conductive coil is preferably discarded therewith.

At a first end a first conductive wire 40 and a second conductive wire 42, both of which are integral with the electrically conductive coil 35, are operatively connected to a controlling circuit 44 having a source knob 45 which, in use, can be turned on to generate a TVEMF. At a second end the wires 40, 42 are connected to at least one ring to facilitate the rotation of the electrically conductive coil 35, preferably a first ring 46 and a second ring 48 respectively. When the control knob 24 is turned on, the culture chamber 32 and the electrically conductive coil 35 are rotated simultaneously. Furthermore, the electrically conductive coil 35 remains affixed to, and encompassing, the culture chamber 32, while at the same time supplying a TVEMF to the cells in the culture chamber 32.

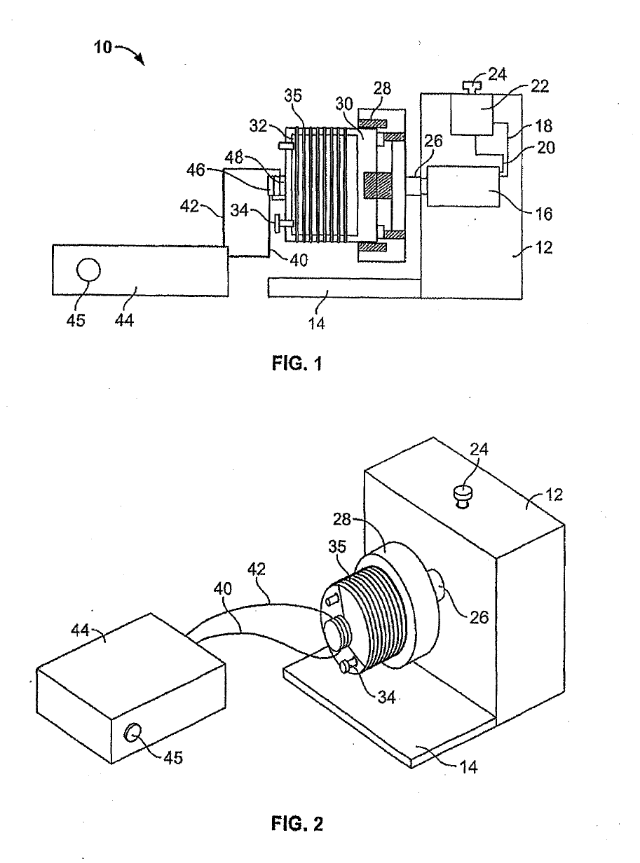

FIG. 2 illustrates a side perspective view of a rotatable bioreactor wound by an electrically conductive coil 35 also depicted in the preferred embodiment of FIG. 1 wherein the electrically conductive coil 35 is wrapped around, and encompassing, a culture chamber 32.

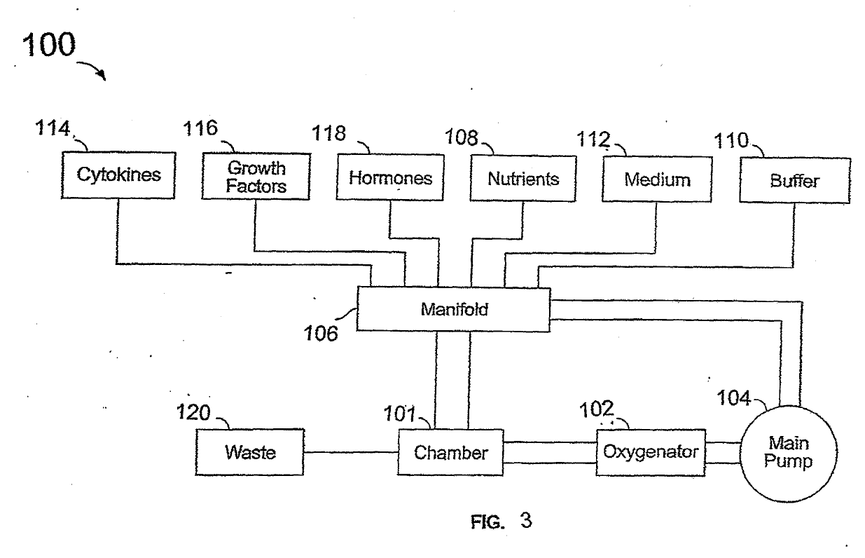

The culture chamber of a rotatable bioreactor may preferably be fitted with a culture medium flow loop preferably for the support of respiratory gas exchange, supply of nutrients, and removal of metabolic waste products from a three-dimensional culture. A preferred embodiment of a culture medium flow loop 100 is illustrated in FIG. 3, having a culture chamber 101, an oxygenator 102, an apparatus for facilitating the directional flow of the culture medium, preferably by the use of a main pump 104, and a supply manifold 106 for the selective input of culture medium requirements such as, but not limited to, nutrients 108, buffers 110, fresh culture medium 112, cytokines 114, growth factors 116, and hormones 118. In this preferred embodiment, the main pump 104 provides fresh culture medium from the supply manifold 106 to the oxygenator 102 where the culture medium is oxygenated and passed through the culture chamber 101. The waste in the spent culture medium from the culture chamber 101 is removed, preferably by the main pump 104, and delivered to the waste 120 and the remaining volume of culture medium not removed to the waste 120 is returned to the supply manifold 106 where it may preferably receive a fresh charge of culture medium requirements before recycling by the pump 104 through the oxygenator 102 to the culture chamber 101.

In this preferred embodiment of a culture medium flow loop 100, adjustments are made in response to chemical sensors (not shown) that maintain constant conditions within the culture chamber 101. Controlling carbon dioxide pressures and introducing acids or bases corrects pH. Oxygen, nitrogen, and carbon dioxide are dissolved in a gas exchange system (not shown) in order to support cell respiration. The culture medium flow loop 100 adds oxygen and removes carbon dioxide from a circulating gas capacitance. Although FIG. 3 is one preferred embodiment of a culture medium flow loop that may be used in the present invention, the invention is not intended to be so limited. The input of culture medium requirements such as, but not limited to, oxygen, nutrients, buffers, fresh culture medium, cytokines, growth factors, and hormones into a rotatable bioreactor can also be performed manually, automatically, or by other control means, as can be the control and removal of waste and carbon dioxide. Also, for instance, nutrients, buffer, culture medium, hormones, cytokines, growth factors and other desired components may be combined for instance into one solution, so that the manifold need only control the addition of one solution to the culture chamber.

Another preferred bioreactor of the present invention relates to a rotatable bioreactor comprising a rotatable perfusable culture chamber and a TVEMF source operatively connected to the rotatable perfusable culture chamber.

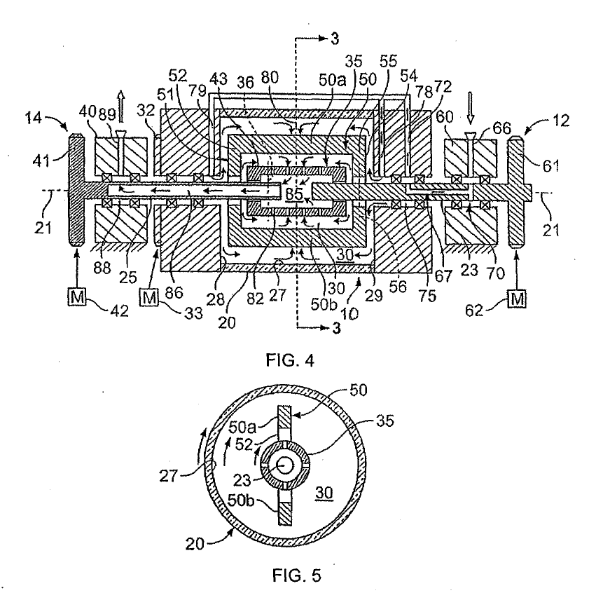

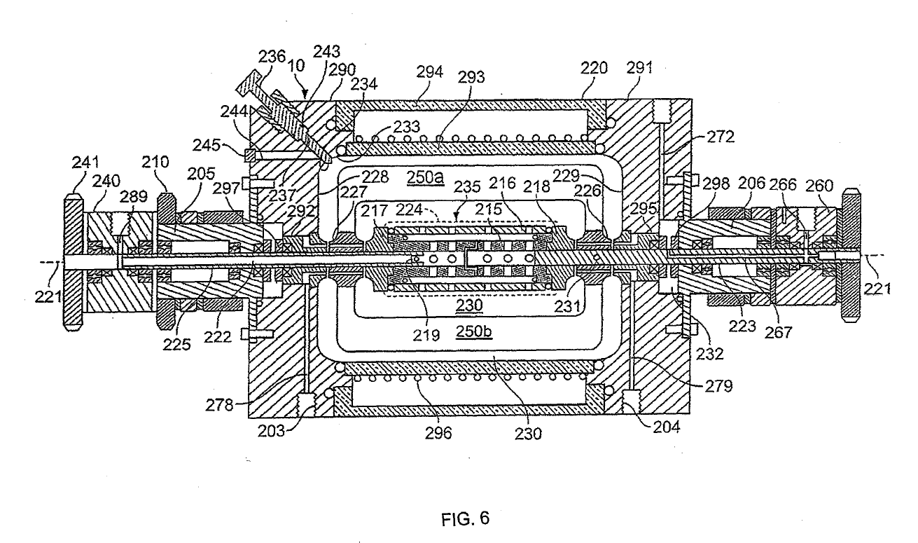

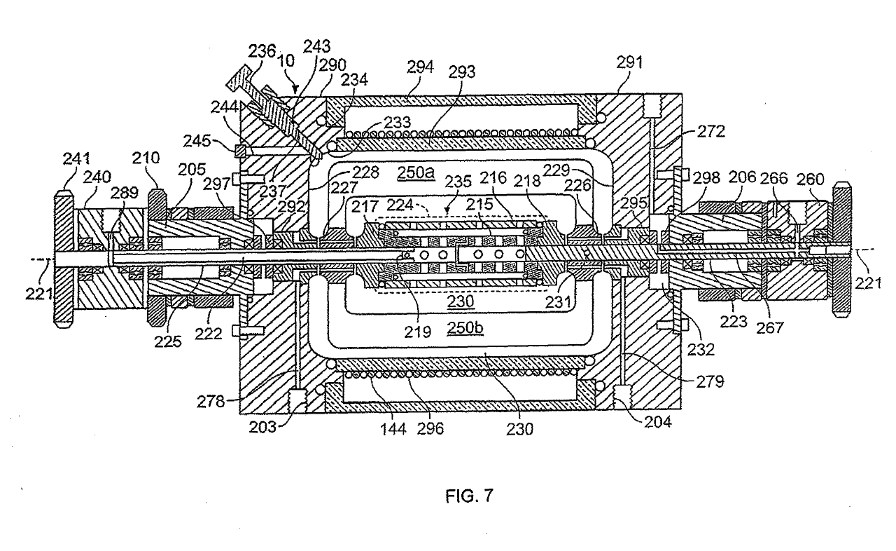

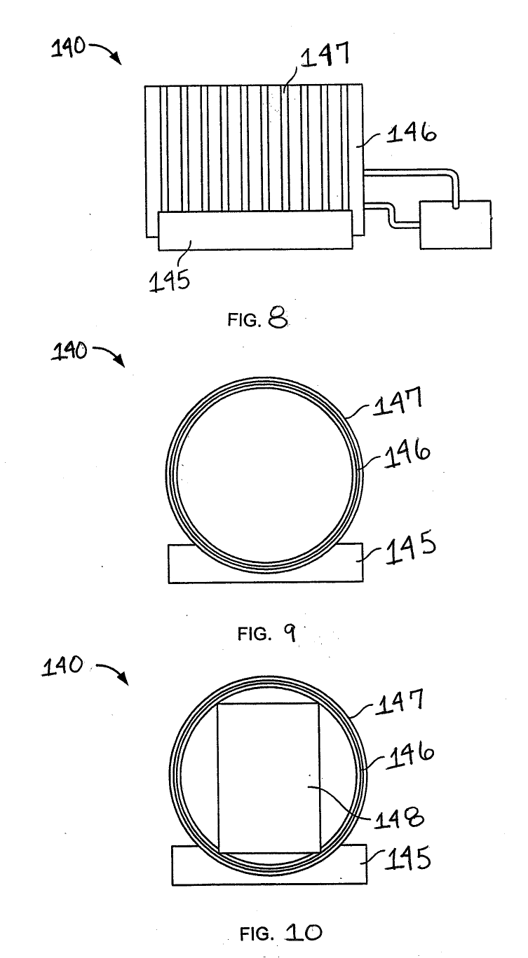

FIGS. 4 and 5 illustrate a preferred embodiment of a rotatable bioreactor 10 with a perfused culture chamber that can be used with an adjacent TVEMF-source (not shown) if the preferred embodiment including exposing the cells to a TVEMF is contemplated in the culture chamber. FIG. 6 is a cross section of a rotatable bioreactor 10 with a perfused culture chamber for use in the present invention in a preferred form with an integral TVEMF-source. FIG. 7 illustrates a rotatable bioreactor 10 with a perfused culture chamber with an integral TVEMF-source. FIGS. 8-10 show an embodiment of an adjacent TVEMF-source for a rotatable bioreactor.

When in use, such a perfused bioreactor provides for the support of respiratory gas exchange in, supply of nutrients in, and removal of metabolic waste products from a three-dimensional culture by the perfusion of culture medium through the rotatable perfusable culture chamber. The term “perfusable,” and similar terms, is intended to mean that the culture medium may be poured over, diffused or permeated through, cells in a perfusable rotatable culture chamber of a rotatable perfused bioreactor. The perfusion may preferably be through a culture medium flow loop as illustrated in FIG. 3, more preferably via direct injection, and most preferably via exchange across a diffusion membrane. In operation, the culture medium is circulated through the three-dimensional culture in the rotatable perfusable culture chamber 19 and preferably around the culture medium flow loop 100, as shown in FIG. 3. In this preferred embodiment of a culture medium flow loop 100, adjustments are made in response to chemical sensors (not shown) that maintain constant conditions within the rotatable perfusable culture chamber 19. Controlling carbon dioxide pressures and introducing acids or bases corrects pH. Oxygen, nitrogen, and carbon dioxide are dissolved in a gas exchange system (not shown) in order to support cell respiration. The culture medium flow loop 1 adds oxygen and removes carbon dioxide from a circulating gas capacitance. Although FIG. 3 is one preferred embodiment of a culture medium flow loop that may be used in the present invention, the invention is not intended to be so limited. The input of culture medium requirements such as, but not limited to, oxygen, nutrients, buffers, fresh culture medium, cytokines, growth factors, and hormones into a rotatable perfused bioreactor can also be performed manually, automatically, or by other control means, as can be the control and removal of waste and carbon dioxide.

FIG. 4 is a cross-sectional side view of a preferred embodiment of a rotatable bioreactor 10 with a perfused culture chamber that is operatively connected to an adjacent TVEMF-source (not shown) having an input end 12 and an output end 14. In FIG. 4, an outer tubular housing 20 rotatably supported for rotation about a horizontal central axis 21 and about an input shaft 23 and an output shaft 25 which are aligned with the central axis 21 (not shown except by dashed line). The outer tubular housing 20 has an interior wall 27, preferably cylindrically shaped, an output transverse end wall 28, and an input traverse end wall 29 that generally define a rotatable perfusable culture chamber 30, preferably cylindrically shaped, preferably elongated. A spur gear 32 is attached to one end of the outer tubular housing 20 and is driven by a motor 33 to rotate the housing 20 about its horizontal central axis 21.

Coaxially disposed about the central axis 21 is an inner filter assembly 35, preferably tubular, that is rotatably mounted on the input shaft 23 and is coupled (as shown by the dashed line 36) to the output shaft 25. The output shaft 25, in turn, is rotatably supported in an output stationary housing 40 and the output shaft 25 has an externally located output spur gear 41 that is connected to a first independent drive motor 42 for rotating the output shaft 25 and the inner filter assembly 35 independently of the outer housing 20. The annular space between the inner filter assembly 35 and the interior wall 27 of the outer tubular housing 20 define the rotatable perfusable culture chamber 30 located about the horizontal axis 21. Intermediate of the outer wall 43 of the inner filter assembly 35 and the interior wall 27 of the outer housing 20 is an intermediate blade member system 50 which preferably includes two lengthwise extending blade members 50a and 50b which are preferably equiangularly spaced from one another about the central axis 21. Each of the blade members 50a and 50b at a first longitudinal end 51 has a second radial arm 52 which is rotatably supported on the output shaft 25 and at a second longitudinal end 54 has a second radial arm 55 which is coupled to the input shaft 23 (shown by dashed line 56). The input shaft 23, in turn, is rotatably mounted in an input stationary housing 60 and the input shaft 23 has an input spur gear 61 that is driven by a second independent drive motor 62 for rotation of the blade member system 50 independent of the rotation of the outer housing 20.

As shown in FIG. 5, the angular rotation of the three sub-assemblies 20, 35 and 50, i.e., the inner filter assembly or member 35, the outer housing 20, and the intermediate blade member system 50, may preferably be at the same angular rate and in the same direction about a horizontal rotational axis and preferably substantially in the same direction about a horizontal axis so that there is little to no relative movement between the three sub-assemblies. This condition of operation obtains a clinostat suspension of microcarrier beads in a fluid culture medium within the rotatable perfusable culture chamber 30 of the rotatable bioreactor 10 without turbulence.

The rotation of the filter 35 can preferably be started and stopped to, for instance, add culture medium, which will cause turbulence on the surface of the filter 35 and keep the surface clean. The blade members 50a and 50b assist cells as they grow to maintain spatial positions in the rotating culture medium. This is particularly helpful for higher density cells, tissues, and tissue-like structures, for instance, bone cells. By rotating the fluid and the outer housing 20, the velocity gradient at the wall boundary layer is nearly eliminated.

The rotatable bioreactor 10 of FIG. 4, in operation provides for culture medium preferably containing fresh nutrients and gases to be input to an input passageway 66 in the input stationary housing 60 and connects to an input longitudinal passageway 67 in the input shaft 23 by virtue of a sealed input rotative coupling 70. The input passageway 67 in the input shaft 23 couples to a radial supply passageway 72 in an end cap of the outer housing 20 by virtue of a sealed input rotative coupling 75. The radial supply passageway 72, in turn, connects to space apart a radially directed input end input passage 78 and output end input passage 79 in the outer housing 20 where the input end input passage 78, and output end input passage 79 are located at opposite ends of the rotatable perfusable culture chamber 30. As shown by the arrows, when culture medium is input at both ends of the rotatable perfusable culture chamber 30, the culture medium moves radially outward toward the interior wall 27 of the outer housing 20 and then moves longitudinally in a horizontal direction toward a midpoint plane generally indicated by a vertically dashed line 80 and then moves radially inwardly toward the outer wall 43 of inner filter assembly 35. Thus the culture medium in the chamber 30 has a generally toroidal type of motion in radial planes on either side of the midpoint plane 80 of the outer housing 20. The inner filter assembly 35 has apertures 82 along its length for exit of culture medium from the rotatable perfusable culture chamber 30 to the interior and, while not illustrated in FIG. 4, preferably there is a lengthwise extending filter cloth located across the apertures 82 that prevents microcarrier bead members in the chamber 30 from exiting through the apertures 82. Spent culture medium in the rotatable perfusable culture chamber 30 thus is passed to the interior 85 of the inner filter assembly 35 and exits via an output longitudinal passageway 86 in the output shaft 25 to an output rotative coupling output 88 in the output stationary housing 40 and to an output passageway 89 preferably to the return of the culture medium flow loop for recharging (not shown).

Turning now to the embodiment in FIG. 6 of a rotatable bioreactor 10 comprising a rotatable perfusable culture chamber 230 and a TVEMF-source operatively connected to the rotatable perfusable culture chamber 230, wherein the TVEMF-source comprises an annular wire heater 296. The annular wire heater 296 is integral with the rotatable perfusable culture chamber 230. The rotatable perfusable culture chamber 230 is preferably transparent, and further comprising an outer housing 220 which includes a first 290 and second 291 transverse end cap member, preferably cylindrically shaped, having facing first 228 and second 229 end surfaces arranged to receive an inner member 293, preferably cylindrical tubular, preferably transparent, and more preferably glass, and an outer tubular member 294, preferably transparent, and preferably glass. Suitable pressure seals are well known in the art and are preferably provided. Between the inner 293 and outer 294 tubular members is an annular wire heater 296 that can preferably be utilized for obtaining the proper incubation temperatures for TVEMF-expansion. The wire heater 296 can also preferably be used as a part of a TVEMF-source that, in use, supplies a TVEMF to the rotatable perfusable culture chamber 230. The first end cap member 290 and second end cap member 291 have inner curved surfaces adjoining the end surfaces 228, 229 for promoting smoother flow of the mixture within the culture chamber 230. The first end cap member 290, and second end cap member 291 have a first central fluid transfer journal member 292 and second central fluid transfer journal member 295, respectively, that are rotatably received respectively on an input shaft 223 and an output shaft 225. Each transfer journal member 294, 295 has a flange to seat in a recessed counter bore in an end cap member 290, 291 and is attached by a first lock washer and ring 297, and second lock washer and ring 298 against longitudinal motion relative to a shaft 223, 225. Each journal member 294, 295 has an intermediate annular recess that is connected to longitudinally extending, circumferentially arranged passages. Each annular recess in a journal member 292, 295 is coupled by a first radially disposed passage 278 and second radially disposed passage 279 in an end cap member 290 and 291, respectively, to first input coupling 203 and second input coupling 204. In operation, culture medium in a radial passage 278 or 279 flows through a first annular recess and the longitudinal passages in a journal member 292 or 295 to permit access to the culture medium through a journal member 292, 295 to each end of the journal member 292, 295 where preferably the access is circumferential about a shaft 223, 225.

Attached to the end cap members 290 and 291 are a first tubular bearing housing 205, and second tubular bearing housing 206 containing ball bearings which relatively support the outer housing 220 on the input 223 and output 225 shafts. The first bearing housing 205 has an attached first sprocket gear 210 for providing a rotative drive for the outer housing 220 in a rotative direction about the input 223 and output 225 shafts and the longitudinal axis 221. The first bearing housing 205, and second bearing housing 206 also preferably have provisions for electrical take out of the annular wire heater 296 and preferably any other sensor, for instance a sensor to detect a change in the location of the cells, preferably and/or a sensor to detect a change in the pH and/or the temperature of the three-dimensional culture.

The inner filter assembly 235 includes inner 215 and outer 216 tubular members having perforations and/or apertures along their lengths and have a first 217 and second 218 inner filter assembly end cap member with perforations. The inner tubular member 215 is preferably constructed in two pieces with an interlocking centrally located coupling section and each piece attached to an end cap 217 or 218. The outer tubular member 216 is preferably mounted between the first 217 and second inner filter assembly end caps.

The end cap members 217, 218 are respectively rotatably supported on the input shaft 223 and the output shaft 225. The inner member 215 is rotatively attached to the output shaft 225 preferably by a pin and an interfitting groove 219. A cloth 224, preferably nylon, with a weave, preferably ten-microns, is disposed over the outer surface of the outer member 216 and is attached at either end, preferably with O-rings. Because the inner member 215 is attached to a slot in the output drive shaft 225, preferably by a coupling pin, the output drive shaft 225 can rotate the inner member 215. The inner member 215 is coupled by the first 217 and second 218 end caps that support the outer member 216. The output shaft 225 is extended through bearings in a first stationary housing 240 and is coupled to a first sprocket gear 241. As illustrated, the output shaft 225 has a tubular bore 222 that extends from a first passageway 289 in the first stationary housing 240 located between seals to the inner member 215 so that, in use, a flow of culture medium can be exited from the inner member 215 through the stationary housing 240.

Between the first 217 and second 218 end caps for the inner member 235 and the journals 292, 295 in the outer housing 220, are a first 227 and second 226 hub for the blade members 250a and 250b. The second hub 226 on the input shaft 223 is coupled to the input shaft 223 by a pin 231 so that the second hub 226 preferably rotates with the input shaft 223. Each hub 227, 226 has axially extending passageways for the transmittal of culture medium through a hub.

The input shaft 223 extends through bearings in the second stationary housing 260 for rotatable support of the input shaft 223. A second longitudinal passageway 267 extends through the input shaft 223 to a location intermediate of retaining washers and rings that are disposed in a second annular recess 232 between the faceplate and the housing 260. A third radial passageway 272 in the second end cap member 291 permits culture medium in the recess to exit from the second end cap member 291. While not shown, the third passageway 272 connects to each of the passages 278 and 279, preferably through piping and a Y joint.

A sample port is shown in FIG. 6, where a first bore 237 extending along a first axis intersects a corner 233 of the chamber 230 and forms a restricted aperture 234. Preferably the bore 237 has a counter bore and a threaded ring at one end to receive a cylindrical valve member 236, preferably threadedly. The valve member 236 protrudes slightly into the interior of the chamber 230, and the valve member 236 comprises a complimentarily formed tip to engage the opening 234 and. Preferably, an O-ring 243 on the valve member 236 provides a seal. A second bore 244 along a second axis intersects the first bore 237 at a location between the O-ring 243 and the opening 234. Preferably an elastomer or plastic stopper 245 closes the second bore 244 that can be entered with a syringe for removing a sample. To remove a sample, the valve member 236 is backed off to access the opening 234 and the bore 244. A syringe can then be used to extract a sample and the opening 234 can be reclosed, and therefore, no outside contamination reaches the interior of the rotatable bioreactor 10.

In operation, culture medium is input to the second port or passageway 266 to the shaft passageway and thence to the first radially disposed 278 and second radially disposed passageways 279 via the third radial passageway 272. In operation, when the culture medium enters the chamber 230 via the longitudinal passages in the journals 292, 294 the culture medium impinges on an end surface 228, 229 of the hubs 227, 226 and is dispersed radially as well as axially through the passageways in the hubs 227, 226. Culture medium passing through the hubs 227, 226 impinges on the end cap members 217, 218 and is dispersed radially. The flow of entry culture medium is thus radially outward away from the longitudinal axis 221 and flows in a toroidal fashion from each end to exit through the polyester cloth 224 and openings in the filter assembly 235 to exit via the passageways 266 and 289. By controlling the rotational speed and direction of rotation of the outer housing 220, chamber 230, and inner filter assembly 235 any desired type of culture medium action can be obtained. Preferably a clinostat operation can be obtained together with a continuous supply of culture medium.

FIG. 7 is a cross-sectional elevated side view of the preferred embodiment of a rotatable bioreactor 10 with a perfusable culture chamber. The preferred embodiment of a rotatable bioreactor 10, illustrated in FIG. 7, shows an integral TVEMF source comprising a wire coil 144 and that is separate from the annular wire heater 296, both of which can be used to deliver a TVEMF to cells in a culture chamber. The embodiment of a rotatable bioreactor 10 illustrated in FIG. 7 is different from the embodiment of a rotatable bioreactor 10 illustrated in FIG. 6 because FIG. 6 only discloses an annular wire heater 296 that can preferably be used as a part of a TVEMF-source.

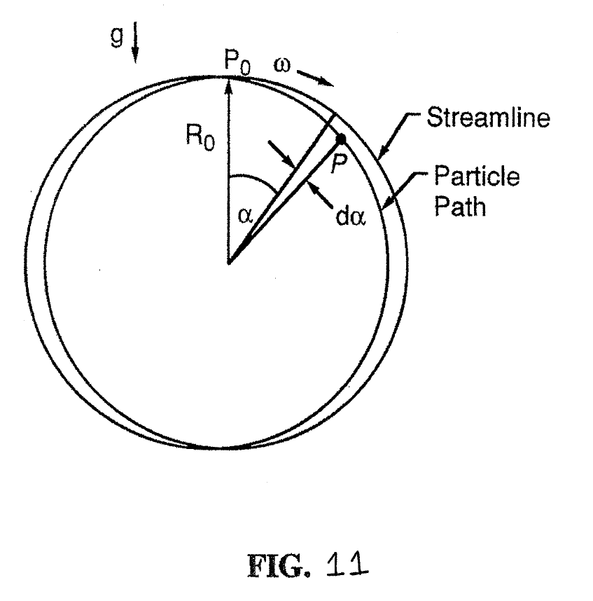

If a TVEMF is not applied using an integral TVEMF-source for instance an annular wire heater 296, as in FIG. 6, or an integral TVEMF-source for instance a wire coil 144 as in FIG. 7, it can be supplied by another preferred TVEMF-source. For instance, FIGS. 8-10 illustrate a preferred embodiment of yet another TVEMF-source comprising a TVEMF-device 140, that may preferably supply a TVEMF to a three-dimensional culture in a rotating rotatable bioreactor which does not have an integral TVEMF-source, for example the rotatable bioreactor illustrated in FIG. 4. Specifically, FIG. 8 is a preferred embodiment of a TVEMF-device 140 that may be operatively connected to a rotatable culture chamber. FIG. 8 is an elevated side perspective of the TVEMF-device 140 which comprises a support base 145, a coil support 146 disposed on the base 145 with a wire coil 147 wrapped around the support 146. FIG. 9 is an elevated front perspective of a TVEMF-device 140 illustrated in FIG. 8. FIG. 10 illustrates the TVEMF-device operatively connected to a rotatable bioreactor 148, so that the rotatable bioreactor 148 in FIG. 10 has an adjacent TVEMF-source. In use, a rotatable bioreactor 148 may preferably be inserted into the coil support 146 which is disposed on a support base 145 and which is wound by a wire coil 147. Since the TVEMF-device 140 is adjacent to the rotatable bioreactor 148, the TVEMF-device 140 can be reused as a TVEMF-source. In addition, since the TVEMF-device 140 is adjacent to the rotatable bioreactor 148, the TVEMF-device 140 can be used to generate a TVEMF in all types of rotatable bioreactors. The present invention also contemplates that the rotatable bioreactor may have a TVEMF-source that comprises at least one loop around the culture chamber, wherein in use, a TVEMF is supplied to the cells in the culture chamber.

As various changes could be made in rotatable bioreactors such as are contemplated in the present invention, without departing from the scope of the invention, it is intended that all matter contained herein be interpreted as illustrative and not limiting.

Other aspects, features, and advantages of the present invention will be apparent from the following description of the preferred embodiments of the invention given for the purpose of disclosure. This invention may be more fully described by the preferred embodiment(s) as hereinafter described, but is not intended to be limited thereto.

### DETAILED DESCRIPTION OF THE INVENTION

The following definitions are meant to aid in the description and understanding of the defined terms in the context of the present invention. The definitions are not meant to limit these terms to less than is described throughout this application, just as definitions relating to the above Figures are not meant to be limited only to a given Figure. In particular, several definitions are included relating to TVEMF—all of the definitions in this regard should be considered to complement each other, and not construed against each other.

As used throughout this application, the term “rotatable bioreactor” refers to a bioreactor that can be rotated about a substantially horizontal axis, horizontal to the plane of the earth, and if the culture chamber is substantially cylindrical, about the culture chambers longitudinal axis. In addition, the rotatable bioreactor is rotated 360 degrees in one direction so that the cells contained therein are suspended in discrete microenvironments with very little, if any, turbulence and low shear stress. A short recess is permitted wherein culture media can, for instance, be refreshed, or samples taken, or for other reasons, without disturbing the suspension of the cells in the rotating bioreactor. The rotatable bioreactor of the present invention, with and without TVEMF, provides a three-dimensional environment wherein the entire volume of the culture chamber is filled so as to provide essentially zero headspace. In addition, the rotating bioreactor essentially mimics a microgravity situation. Preferably, a rotatable bioreactor allows for the exchange of growth medium (preferably with additives) and for oxygenation of the peripheral blood mixture. The rotatable bioreactor provides a mechanism for expanding cells for several days or more. The rotation of the rotatable bioreactor during expansion is preferably at a rate of 5 to 120 rpm, more preferably 10 to 30 rpm, to foster minimal wall collision frequency and intensity so as to maintain at least a portion of the cells underlying characteristics before expansion. The volume of the culture chamber of a rotatable bioreactor is preferably from about 15 ml to about 2 L. See for instance FIGS. 1 and 2 herein for examples (not meant to be limiting) of a rotatable bioreactor of the present invention.

A rotating bioreactor can be made a rotating TVEMF bioreactor with the addition of time varying electromagnetic force (“TVEMF”). Without being bound by theory, it is thought that the TVEMF provides the expanded cells with unique phenotypic characteristics such that the genetic expression of the cells is affected by the TVEMF exposure. The TVEMF exposure is such that it is passed through or otherwise exposed to the cells, the cells thus undergoing TVEMF-expansion. As used throughout this application, the term “exposing,” and similar terms, refers to the process of supplying a TVEMF to cells contained in the interior portion of the culture chamber of a rotatable bioreactor. The TVEMF of the present invention has a slew rate of at least about 10 kiloGauss/sec; a magnetic cycle period of at least about 0.01 Hertz, a magnetic field active duty of at least about 0.01 percent of the cycle period wherein the magnetic active field duty is defined as when the TVEMF source emits the TVEMF and a peak magnetic amplitude having an absolute value of at least about 0.1 Gauss.

The preferred embodiment of a rotatable bioreactor with the application of TVEMF is subject to almost any infinite number of design variations and is considered to apply as long as it can produce a magnetic slew rate (either rising or falling, or both rising and falling) of at least about 10 kiloGauss/sec. For instance, one variation of the rotatable bioreactor with TVEMF is that it is configured to exhibit a slew rate (either rising or falling, or both rising and falling) being between about 25 to about 1000 kiloGauss/sec. Another variation of the rotatable bioreactor with TVEMF is that it is configured to exhibit a magnetic cycle period between about 0.01-1000 Hertz. Yet another variation is that the rotatable bioreactor with TVEMF is that it is configured to exhibit a magnetic field active duty between about 0.01 to 50 (preferably 0.01 to 2) percent of the cycle period wherein the magnetic active field duty defined as when the TVEMF source emits the magnetic field. Still yet another variation is that the rotatable bioreactor with TVEMF is configured to exhibit a magnetic inactive duty being between about 50 to 99.99 (preferably 98 to 99.99) percent of the cycle period wherein the magnetic field inactive duty defined as when the TVEMF source does not emit the magnetic field. Even yet another variation is that the rotatable bioreactor with TVEMF is configured to exhibit can be configured to exhibit a peak magnetic amplitude being between about −20 to +20 Gauss. One variation in the design of the TVEMF source is that it can be configured to exhibit an electrical current slew rate (either rising or falling, or both rising and falling) between about 10 to about 1000 Amperes/sec. Another variation of the TVEMF source is that the output current of the electric pulse train outputted from the controlling circuit can be configured to exhibit a falling slew rate being between about 10 to about 1000 Amperes/sec. Yet another variation of the TVEMF source is that it can be configured to the output the electric pulse train to exhibit an electrical cycle period being between about 0.01-100 Hertz. Still another variation of the TVEMF source is that the output current of the electric pulse train outputted from the TVEMF source can be configured to exhibit an electrical active period between about 0.01 to 50 (preferably 0.01 to 2) percent of the electrical cycle period wherein the electrical active period defined as when the output current is outputted. Yet another variation of the TVEMF source is that the output current of the electric pulse train outputted from the TVEMF source can be configured to exhibit an electrical inactive period between 50 to 99.99 (preferably 98 to 99.99) percent of the electrical cycle period wherein the electrical inactive period defined as when the output current is not outputted. Still yet another variation of the TVEMF source is that the output current of the electric pulse train outputted from the controlling circuit can be configured to exhibit a peak voltage amplitude being between about −5 to +5 Volts and to exhibit a peak current amplitude being between about −5 to +5 kiloAmps. Even yet another variation of the TVEMF source is that the output current can be configured to exhibit a rising electrical current slew rate between about 10 to about 1000 Amperes/sec and to exhibit a falling electrical slew rate being between about 10 to about 1000 Amperes/sec. Still another variation of the TVEMF source is that the electrical cycle period can be configured to be between about 0.01-100 Hertz. Yet another variation of the TVEMF source is that the electrical active period can be configured to be between about 0.01 to 2 percent of the electrical cycle period and that the electrical inactive period can be configured to be between 98 to 99.99 percent of the electrical cycle period. Even yet another variation of the TVEMF source is that the peak voltage amplitude can be configured to be between about 1 to 10 Volts. Still yet another variation of the TVEMF source is that the peak current amplitude can be configured to be being between about 1 to 10 Amps.

As used throughout this application, the term “cells” may refer to a cell in any form, for example, individual cells, tissue, cell aggregates, cells pre-attached to cell attachment substrates for instance microcarrier beads, tissue-like structures, or intact tissue resections. The cells that are preferably used in this invention are from mammalian tissue, preferably human.

As used throughout this application, the term “mesenchymal stem cell” and similar terms refers to mesenchymal cells also known as mesenchymal stem cells or marrow stromal cells are stem cells that can differentiate into osteoblasts, chondrocytes, myocytes and adipocytes. Cells that mesenchymal stem cells may differentiate into during the differentiating step or this invention may include but are not limited to osteoblasts, chondrocytes, myocytes, adipocytes, neuronal cells, beta-pancreatic islet cells. Mesenchymal stem cells differentiate from colony-forming unit-fibroblast (CFU-F). Mesenchymal stem cells cultured without serum in the presence of transformation growth factor (TGF) will differentiate into chondrocytes while Mesenchymal stem cells cultured in serum with ascorbic acid and dexamethasone will differentiate into osteoblasts.

As used throughout this application, the term “mesenchymal stem cell product” and similar terms refers to cells removed from a bioreactor after expansion of mesenchymal stem cells has occurred in a three-dimensional culture in a bioreactor rotated about a substantially horizontal axis. Mesenchymal stem cell product preferably comprises at least one differentiated cell. Cells in a mesenchymal stem cell product may include, but are not limited to, mesenchymal stem cells, osteoblasts, chondrocytes, myocytes, adipocytes, neuronal cells, beta-pancreatic islet cells, and other cells that are partially or completely differentiated from mesenchymal stem cells.

As used throughout this application, the term “biogel” and similar terms refers to a pharmaceutically acceptable substance that can be combined with a mesenchymal stem cell product for delivery to a mammal. Preferred embodiments of a biogel include, but are not limited to, hydrogel polymer, polymerized polyethylene glycol diacrylate, polylactic acid, polyglycolic acid, and polymerized polyethylene glycol dimethylacrylate. In a preferred embodiment of this invention, the biogel can be mixed with the mesenchymal stem cell product to make what is being referred to as a cellular mixture.

As used throughout this application, the term “mesenchymal stem cell mixture” and similar terms, refers to a mixture of cells, preferably with another substance including, but not limited to, culture medium (with or without additives), plasma, buffer, and preservatives. The cell mixture may also solely comprise cells. The mesenchymal stem cell mixture is placed in the culture chamber of the rotatable bioreactor for expansion therein.

As used throughout this application, the term “three-dimensional culture” refers to the cells in the culture chamber during the process of cell expansion and/or differentiation (while the bioreactor is in use) in the culture chamber. Three-dimensional tissue and/or tissue-like structures can also develop from the cells and be sustained and further expanded and/or differentiated in the three-dimensional culture.

As used throughout this application, the term “expanded cells” refers to mesenchymal stem cells increased in number (i.e. concentration), at least one cell is differentiated, and/or the cells are cultured after being placed in a rotatable bioreactor. “TVEMF expanded cells,” and similar terms, refers to cells TVEMF-expanded in a rotatable bioreactor wherein the cells are increased in number, at least one cell is differentiated, and/or the cells are cultured in the rotating rotatable bioreactor and subjected to a TVEMF. The increase in number of cells is the result of cell replication in the bioreactor, so that the total number of cells in the bioreactor increases. The increase in number of cells is expressly not due to a simple reduction in volume of fluid, for instance, reducing the volume from 70 ml to 10 ml and thereby increasing the number of cells per ml. By increasing in number it is intended that the cells replicate (and thereby grow in number). Preferably the cells are expanded to more than seven times the number placed in the culture chamber of the rotatable bioreactor before the expansion of the mesenchymal stem cells.

In a preferred embodiment, the number of cells expanded not important. In such an embodiment, it is contemplated that by culturing the cells in the rotating bioreactor (with or without TVEMF), the cells will have enhanced repairing, regenerating, andor reconstructing capabilities. For instance, the cells may have enhanced tissue repairing characteristics or tissue function repairing characteristics by being cultured in the rotating bioreactor. If the preference is to culture the cells then a user may not focus on the number of cells expanded. For instance, if the culture in the rotating bioreactor is the focus of the method, then zero additional cells, less than the number that were placed in the rotating bioreactor, and at least one more than number that were placed in the bioreactor may all be acceptable numbers.

In another preferred embodiment, at least one mesenchymal stem cell is differentiated. It is not necessary the cell be terminally differentiated, but is at least partially differentiated. Moreover, the present invention contemplates a combination of expanded cells i.e. increased in number, cultured, and/or differentiated. The ultimate product from an expansion (with or without TVEMF) is referred to as a mesenchymal stem cell product. The term “expansion” may also include growth in the size of tissue(s), tissue-like structures, and/or cell aggregates, as well as an increase in the number of cells in a culture container. The term “expanding” refers to the process of expansion while cells are in the culture chamber of a bioreactor. Other aspects of expansion may also provide the exceptional characteristics of the cells of the present invention, which is why the number of expanded cells may not be the focus of the expansion process, but rather, the three-dimensional culture environment in the rotating system may be the focus. Because the cells are expanded, or TVEMF-expanded, an essentially quiescent environment that provides for collocation of the discrete suspension material and mesenchymal stem cells, essentially no relative motion of said culture medium with respect to the boundaries of the chamber, and freedom for three dimensional spatial orientation of assemblies formed by the culturing of the cells, the cells expanded therein exhibit a unique genetic expression including the up or down regulation of genes to maintain the cells in suspension in the three-dimensional environment. Not to be bound by theory, but it is expected that the addition of a TVEMF may affect additional properties of mesenchymal stem cells during TVEMF-expansion, for instance up-regulation of genes promoting growth, or down regulation of genes preventing growth. It is also contemplated that before expansion, the cells may preferably be cultured in a two-dimensional or preferably in a three-dimensional system for a preferred amount of time before placing the cells in the rotating system for expansion.

As used throughout this application, the term “culture medium” and similar terms, refers to a liquid comprising, but not limited to, growth medium and nutrients, which is meant for the sustenance of cells over time. The culture medium may be enriched with any of the following, but is not limited thereto; growth medium, buffers, growth factors, hormones, and cytokines. The culture medium is supplied to the cells for suspension within the culture chamber of the rotatable (preferably TVEMF) bioreactor and to support expansion and/or differentiation. The culture medium may preferably be mixed with the cells before being added to the culture chamber of the rotatable bioreactor thus making a preferred cell mixture, or may preferably be added to the culture chamber before the cells are added thereby mixing the culture medium and the cells in the rotatable bioreactor.

The combination of culture medium and the cells is referred to as a “three-dimensional culture” when located in the rotatable bioreactor and when the expansion process has begun, and/or after TVEMF has been delivered thereto. The culture medium may preferably be enriched and/or refreshed during expansion and/or differentiation as needed. Waste contained in the culture medium, as well as culture medium itself, may preferably be removed from the three-dimensional culture during expansion and/or differentiation as needed. Waste contained in the spent culture medium can be, but is not limited to, metabolic waste, dead cells, and other toxic debris. The culture medium can preferably be enriched with oxygen and preferably has oxygen, carbon dioxide, and nitrogen carrying capabilities.

As used throughout this applications, the term, “placing,” and similar terms, refers to the process of suspending cells in culture medium before adding the cell mixture to the rotatable bioreactor. The term “placing,” may also refer to adding cells to culture medium that is already present in the rotatable bioreactor. Cells can be placed into the rotatable TVEMF bioreactor along with cell attachment substrates such as microcarrier beads.

As used throughout this application, the term “cell-to-cell geometry” refers to the geometry of cells including the spacing, distance between, and physical relationship of the cells relative to one another. For instance, expanded cells, including those of tissues, cell aggregates, and tissue-like structures, of this invention stay in relation to each other as in a natural three-dimensional setting, the tissue in which the cells naturally occur in vivo; the mammalian body. The expanded cells are within the bounds of natural spacing between cells, in contrast to for instance two-dimensional expansion chambers, where such spacing is not preserved.

As used throughout this application, the term “cell-to-cell support” refers to the support one cell provides to an adjacent cell. For instance, tissues, cell aggregates, tissue-like structures, and cells maintain interactions such as chemical, hormonal, neural (where applicable/appropriate) with other cells in the body. In the present invention, these interactions are maintained within normal functioning parameters, meaning they do not for instance begin to send toxic or damaging signals to other cells (unless such would be done in the natural cellular and tissue environment).

As used throughout this application, the term “three-dimensional geometry” refers to the geometry of cells in a three-dimensional state (same as or very similar to their natural state), as opposed to two-dimensional geometry for instance as found in cells grown in a Petri dish, where the cells become flattened and/or stretched. Not to be bound by theory, but the three-dimensional geometry of the cells is maintained, supported, and preserved such that the cell can develop into three-dimensional cell aggregates, tissues and/or tissue-like structures in the three-dimensional culture of the rotatable bioreactor.

For each of the above three definitions, relating to maintenance of “cell-to-cell support” and “cell-to-cell geometry” and “three-dimensional geometry” of the cells of the present invention, the term “essentially the same” and “substantially the same,” means that natural geometry and support are provided in expansion and/or differentiation, so that the cells are not changed in such a way as to be for instance dysfunctional, toxic or harmful to other cells. Rather, the cells of the present invention, during and after expansion and/or differentiation, mimic the in vivo situation.

Throughout this application, reference to the repair of tissue, treatment of disease or condition, and/or reconstruction are not meant to be exclusive but rather relate to the objective of overall tissue repair where improvement in tissue results from administration of a mesenchymal stem cell product as discussed herein. While the present invention is directed in part to diseases or conditions that are symptomatic, and possibly life-threatening, the present invention is also meant to include treatment of minor repair, and even prevention/prophylaxis of disease/condition by early introduction of expanded mesenchymal stem cells, before symptoms or problems in the mammal's (preferably human's) health are notice. Moreover, the process and products of the present invention may also be used for the research of a disease or condition.

Other statements referring to the above-defined terms or other terms used throughout this application are not meant to be limited by the above definitions, and may contribute to the definitions. Information relating to various aspects of this invention is provided throughout this application, and is not meant to be limited only to the section to which it is contained, but is meant to contribute to an understanding of the invention as a whole.

This invention may be more fully described by the preferred embodiment(s) as hereinafter described, but is not intended to be limited thereto.

### Operational Method

### Process of Making a Mesenchymal Stem Cell Mixture

The term “collecting” and similar terms means the acquisition of a mesenchymal stem cell. Preferably, mesenchymal stem cells are collected from an individual mammal, more preferably a human mammal, prior to being placed in the bioreactor culture chamber. Mesenchymal stem cells may also be pooled from more than one mammal, purchased from a commercial source, or otherwise acquired for instance by acquiring colony-forming-unit-fibroblast and causing colony-forming-unit-fibroblast (or less differentiated cells such as stem cells) to differentiate into mesenchymal stem cells. The preferred source of mesenchymal stem cells of the present invention is bone marrow, more preferably human bone marrow. However, other sources of mesenchymal stem cells may be used, for instance from umbilical cord blood.

As discussed throughout the application, various growth factors and/or other chemicals may be added to the three-dimensional culture prior to and/or during the expanding and/or differentiating step(s). Preferably, the mesenchymal stem cells of the present application are differentiated into one or more differentiated cell types, to facilitate use of the resultant mesenchymal stem cell product in a mammal, preferably a human. For instance, the addition of ascorbic acid, inorganic phosphate and dexamethasone during the differentiating step in the bioreactor will cause mesenchymal stem cells to differentiate into osteoblasts. Also for instance, the addition of transformation growth factor, specifically bone morphogenetic protein, during the differenting step will cause mesenchymal stem cells to differentiate into chondrocytes.

### Operational Method

### Expansion

The present invention is directed in part to a process for preparing a mesenchymal stem cell product in a three-dimensional culture. The process comprises the steps of: a. collecting a mesenchymal stem cell; b. placing the mesenchymal stem cell and a culture medium in a culture chamber of a bioreactor; c. expanding the mesenchymal stem cell in the culture chamber of the bioreactor; and e. removing the cell from the culture chamber of the bioreactor to prepare a mesenchymal stem cell product. Preferably, the expanding step provides expanded mesenchymal stem cells in a number that is at least seven times greater than the number of stem cells placed in the culture chamber of the rotatable bioreactor. Preferably, the expanding step comprises an increase in number and differentiation at the same time. Alternatively, in another preferred embodiment, the expanding step may not include an increase in number or differentiation. In another preferred embodiment, there may not be an increase in number but instead only differentiation. In an additional preferred embodiment, the expanding step may include both culture, an increase in the number of cells, and differentiation.

Preferably, the differentiating step provides for at least 10% of mesenchymal stem cells in the bioreactor to be differentiated (either “partially differentiated”, meaning differentiated to a cell that may be further differentiated, or “completely differentiated”, meaning differentiated to a cell that will not differentiate further into a more differentiated cell). First expanding and then differentiating the mesenchymal stem cells is preferred to maximize the number of mesenchymal stem cells and then later desired differentiated cells that may be produced from the increased number of mesenchymal stem cells during the cells' time in the bioreactor. Cells that mesenchymal stem cells may differentiate into during the differentiating step include but are not limited to osteoblasts, chondrocytes, myocytes, adipocytes, neuronal cells, beta-pancreatic islet cells. Preferably, the bioreactor is a TVEMF bioreactor that, when in use, provides a time varying electromagnetic field.

In use, the rotation of a bioreactor (TVEMF or otherwise) provides a stabilized culture environment into which cells may be introduced, suspended, maintained, and expanded with improved retention of delicate three-dimensional structural integrity by simultaneously minimizing the fluid shear stress, providing three-dimensional freedom for cell and substrate spatial orientation, and increasing localization of cells in a particular spatial region for the duration of the expansion (hereinafter referred to as “three criteria”). The rotating TVEMF-bioreactor also provides these three criteria, and at the same time, exposes the cells to a TVEMF. Of particular interest to the present invention is the dimension of the culture chamber, the sedimentation rate of the cells, the rotation rate, the external gravitational field, and the TVEMF.

The stabilized culture environment referred to in the operation of present invention is that condition in the culture medium, particularly the fluid velocity gradients, prior to introduction of cells, which will support a nearly uniform suspension of cells upon their introduction thereby creating a three-dimensional culture upon addition of the cells. In a preferred embodiment, the culture medium is initially stabilized into a near solid body horizontal rotation 360 degrees about an axis within the confines of a similarly rotating chamber wall of a rotatable bioreactor. The rotating continues in the same direction about the axis. The chamber walls are set in motion relative to the culture medium so as to initially introduce essentially no fluid stress shear field therein. Cells, and preferably cell attachment substrates such as beads, are introduced to, and move through, the culture medium in the stabilized culture environment thus creating a three-dimensional culture. The cells move under the influence of gravity, centrifugal, and coriolus forces, and the presence of cells within the culture medium of the three-dimensional culture induces secondary effects to the culture medium. The motion of the culture medium with respect to the culture chamber, fluid shear stress, and other fluid motions, is due to the presence of these cells within the culture medium.

In most cases the cells with which the stabilized culture environment is primed sediment at a slow rate preferably under 0.1 centimeter per second. It is therefore possible, at this early stage of the three-dimensional culture, to select from a broad range of rotational rates (preferably of from about 2 to about 30 RPM) and chamber diameters (preferably of from about 0.5 to about 36 inches). Preferably, the slowest rotational rate is advantageous because it minimizes equipment wear and other logistics associated with handling the three-dimensional culture. The preferred speed of the present invention is of from 5 to 120 RPM, and more preferably from 10 to 30 RPM.

Not to be bound by theory, rotation about a substantially horizontal axis with respect to the external gravity vector at an angular rate optimizes the orbital path of cells suspended within the three-dimensional culture. The progress of the three-dimensional culture is preferably assessed by a visual, manual, or automatic determination. An increase in the density of cells may require appropriate adjustment of the rotation speed in order to optimize the particular paths. An increase in density is related to an increase in the number of cells in the culture chamber. The rotation of the culture chamber optimally controls collision frequencies, collision intensities, and localization of the cells in relation to other cells and also the limiting boundaries of the culture chamber of the rotatable bioreactor. In order to control the rotation, if the cells are observed to excessively distort inwards on the downward side and outwards on the upwards side then the revolutions per minute (“RPM”) may preferably be increased. If the cells are observed to centrifugate excessively to the outer walls then the RPM may preferably be reduced. Optimally, the zero-head space of the three-dimensional culture provides a space wherein cells may preferably be distributed throughout the volume of culture medium effectively utilizing the full culture chamber capacity.

The cell sedimentation rate and the external gravitations field place a lower limit on the fluid shear stress obtainable, even within the operating range of the present invention, due to gravitationally induced drift of the cells through the culture medium of the three-dimensional culture. Calculations and measurements place this minimum fluid shear stress very nearly to that resulting from the cells' terminal sedimentation velocity (through the culture medium) for the external gravity field strength. Centrifugal and coriolis induced motion [classical angular kinematics provide the following equation relating the Coriolis force to an object's mass (m), its velocity in a rotating frame (vᵣ) and the angular velocity of the rotating frame of reference (□): F_{Coriolis}=−2 m (w×vᵣ)] along with secondary effects due to cell and culture medium interactions, act to further degrade the fluid shear stress level as the cells expand.

Not to be bound by theory, but an environment that is substantially similar to microgravity may be obtained in the rotating bioreactor. In order to obtain the minimal fluid shear stress level it is preferable that the culture chamber be rotated at substantially the same rate as the culture medium. Not to be bound by theory, but this minimizes the fluid velocity gradient induced upon the three-dimensional culture. It is advantageous to control the rate of expansion in order to maintain the cell density (and associated sedimentation rate) within a range for which the rate of expansion is able to satisfy the three criteria. In addition, transient disruptions of the expansion process are permitted and tolerated for, among other reasons, logistical purposes during initial system priming, sample acquisition, system maintenance, and culture termination.

Rotating cells about an axis substantially perpendicular to gravity can produce a variety of sedimentation rates, all of which according to the present invention remain spatially localized in distinct regions for extended periods of time ranging from seconds (when sedimentation characteristics are large) to hours or days (when sedimentation differences are small). Not to be bound by theory, but this allows these cells sufficient time to interact and associate as necessary with each other in a three-dimensional culture. Preferably, cells undergo expansion for at least 4 days, more preferably from about 7 days to about 14 days, most preferably from about 7 days to about 10 days, even more preferably about 7 days. Expansion may continue in a bioreactor (TVEMF or otherwise) for up to 160 days. While expansion may occur for even longer than 160 days, such a lengthy expansion is not a preferred embodiment of the present invention. Preferably, expansion may continue in a rotatable bioreactor to produce a number of cells that is at least 7 times the original number of cells that were placed in the rotatable bioreactor.

Culture chamber dimensions also influence the path of cells in the three-dimensional culture of the present invention. A culture chamber diameter is preferably chosen which has the appropriate volume, preferably of from about 15 ml to about 2 L for the intended three-dimensional culture and which will allow a sufficient seeding density of cells. Not to be bound by theory, but the outward cells drift due to centrifugal force is exaggerated at higher culture chamber radii and for rapidly sedimenting cells.

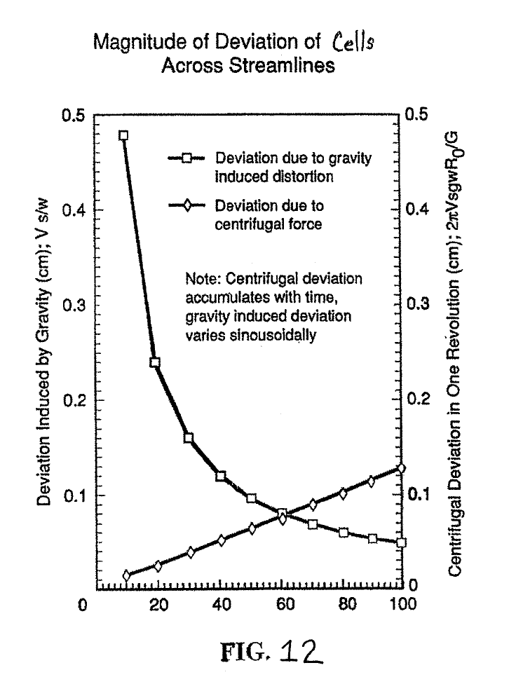

The path of the cells in the three-dimensional culture has been analytically calculated incorporating the cell motion resulting from gravity, centrifugation, and coriolus effects. A computer simulation of these governing equations allows the operator to model the process and select parameters acceptable (or optimal) for the particular planned three-dimensional culture. FIG. 11 shows the typical shape of the cell orbit as observed from the external (non-rotating) reference frame. FIG. 12 is a graph of the radial deviation of a cell from the ideal circular streamline plotted as a function of RPM (for a typical cell sedimenting at 0.5 cm per second terminal velocity). This graph (FIG. 12) shows the decreasing amplitude of the sinusoidally varying radial cells deviation as induced by gravitational sedimentation. FIG. 12 also shows increasing radial cell deviation (per revolution) due to centrifugation as RPM is increased. These opposing constraints influence carefully choosing the optimal RPM to preferably minimize cell impact with, or accumulation at, the chamber walls. A family of curves is generated which is increasingly restrictive, in terms of workable RPM selections, as the external gravity field strength is increased or the cell sedimentation rate is increased. This family of curves, or preferably the computer model which solves these governing orbit equations, is preferably utilized to select the optimal RPM and chamber dimensions for the expansion of cells of a given sedimentation rate in a given external gravity field strength. Not to be bound by theory, but as a typical three-dimensional culture is expanded the number of cells and therefore the cell density effects the sedimentation rate, and therefore, the rotation rate may preferably be adjusted to optimize the same.

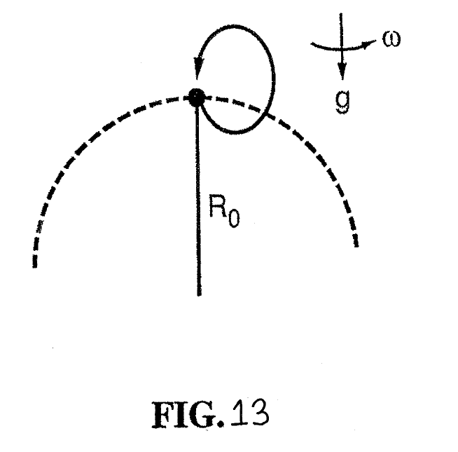

In the three-dimensional culture, the cell orbit (FIG. 11) from the rotating reference frame of the culture medium is seen to move in a nearly circular path under the influence of the rotating gravity vector (FIG. 13). Not to be bound by theory, but the two pseudo forces, coriolis and centrifugal, result from the rotating (accelerated) reference frame and cause distortion of the otherwise nearly circular path. Higher gravity levels and higher cell sedimentation rates produce larger radius circular paths which correspond to larger trajectory deviations from the ideal circular orbit as seen in the non-rotating reference frame. In the rotating reference frame it is thought, not to be bound by theory, that cells of differing sedimentation rates will remain spatially localized near each other for long periods of time with greatly reduced net cumulative separation than if the gravity vector were not rotated; the cells are sedimenting, but in a small circle (as observed in the rotating reference frame). Thus, in operation the present invention provides cells of differing sedimentation properties with sufficient time to interact mechanically and through soluble chemical signals thereby effecting their cell-to-cell interactions including geometry and support. In operation, the present invention provides for sedimentation rates of preferably from about 0 cm/second up to 10 cm/second.

Furthermore, in operation the culture chamber of the present invention has at least one aperture preferably for the input of fresh culture medium and a cell mixture and the removal of a volume of spent culture medium containing metabolic waste, but not limited thereto. Preferably, the exchange of culture medium can also be via a culture medium loop wherein fresh or recycled culture medium may be moved within the culture chamber preferably at a rate sufficient to support metabolic gas exchange, nutrient delivery, and metabolic waste product removal. This may slightly degrade the otherwise quiescent three-dimensional culture. It is preferable, therefore, to introduce a mechanism for the support of preferred components including, but not limited to, respiratory gas exchange, nutrient delivery, growth factor delivery to the culture medium of the three-dimensional culture, and also a mechanism for metabolic waste product removal in order to provide a long term three-dimensional culture able to support significant metabolic loads for periods of hours to months.

It is expected that expansion in a rotating bioreactor provides a unique environment that effects the cell phenotype, as gauged by RNA expression levels. The cells adapt to the unique three-dimensional environment in which they are suspended. Cells expanded in the three-dimensional environment of a rotating bioreactor express different gene expression patterns, and therefore, different membrane and surface protein configurations, and different cytoskeletal details. This feature of the cells is a result of the three-dimensional environment in which the cells are suspended and expanded, referred to as genetic expression modified. It is expected that the cell exposure to TVEMF in a TVEMF-bioreactor provides even more exceptional characteristics to the expanding cell than those detected by rotation alone.

During the time that the cells are in the rotating bioreactor (with or without TVEMF), they are preferably fed nutrients and fresh media, exposed to hormones, cytokines, and/or growth factors (preferably G-CSF); and toxic materials are removed.

Preferably, expansion is carried out in a rotating bioreactor at a temperature of about 26 C to about 41 C, and more preferably, at a temperature of about 37 C.

One method of monitoring the overall expansion of cells undergoing expansion is by visual inspection. Once the bioreactor begins to rotate, and in a preferred embodiment the TVEMF is applied, the cells that are distributed throughout the full volume of media preferably cluster in the center of the bioreactor vessel as they become greater in number (denser), with the medium surrounding the colored cluster of cells. Oxygenation and other nutrient additions often do not cloud the ability to visualize the cell cluster through a visualization (typically clear plastic) window built into the bioreactor. Formation of the cluster is important for helping the stem cells maintain their three-dimensional geometry and cell-to-cell support and cell-to-cell geometry; if the cluster appears to scatter and cells begin to contact the wall of the bioreactor vessel, the rotational speed is increased (manually or automatically) so that the centralized cluster of cells may form again. A measurement of the visible diameter of the cell cluster taken soon after formation may be compared with later cluster diameters, to indicate the approximate number increase in cells in the bioreactor. Measurement of the increase in the number of cells during expansion may also be taken in a number of ways, as known in the art. An automatic sensor could also be included in the bioreactor to monitor and measure the increase in cluster size.

The expansion process may be carefully monitored, for instance by a laboratory expert, who will check cell cluster formation to ensure the cells remain clustered inside the bioreactor and will increase the rotation of the bioreactor when the cell cluster begins to scatter. An automatic system for monitoring the cell cluster and viscosity of the mesenchymal stem cell mixture inside the bioreactor may also monitor the cell clusters. A change in the viscosity of the cell cluster may become apparent about 2 days after beginning the expansion process, and the rotational speed of the bioreactor may be increased around that time. The bioreactor speed may vary throughout expansion. Preferably, the rotational speed is timely adjusted so that the cells undergoing expansion do not contact the sides of the rotating bioreactor vessel.

Also, the laboratory expert may, for instance once a day, or once every two days, manually (for instance with a syringe) insert fresh media and preferably other desired additives such as nutrients and growth factors, as discussed above, into the bioreactor, and draw off the old media containing cell wastes and toxins. Also, fresh media and other additives may be automatically pumped into the bioreactor during expansion, and wastes automatically removed.

Mesenchymal stem cells may increase to at least seven times their original number about 7 to about 14 days after being placed in the bioreactor and expanded. Preferably, the expansion lasts about 7 to 10 days, and more preferably about 7 days. Measurement of the number of stem cells does not need to be taken during expansion therefore. As indicated above and throughout this application, expanded mesenchymal cells of the present invention retain some of the same three-dimensional geometry and cell-to-cell support and cell-to-cell geometry as naturally-occurring, non-expanded mesenchymal cells due to the essentially non-turbulent and low shear stress culture regime. The expanded mesenchymal cell retains fundamental properties of the non-expanded mesenchymal cells. The gentle free drifting of the cells through soluble molecular species which control cell function and are substrates and products of cell metabolism allows the rotating bioreactor systems to produce a unique living product cell in terms of transcribed RNA pattern coding for multiple cell structural and functional proteins and cell sub organelles.

Another embodiment of the present invention relates to an ex vivo mammalian peripheral blood stem cell composition that functions to assist a body system or tissue to repair, replenish and regenerate tissue, for example, the tissues described throughout this application. The composition comprises expanded peripheral blood stem cells, preferably with TVEMF. The peripheral blood cells in the composition are preferably expanded to at least seven times the number that were placed in the culture chamber of the rotatable bioreactor. For instance, preferably, if a number X of peripheral blood stem cells was placed in a certain volume into a bioreactor, then after expansion, the number of peripheral blood stem cells from that same volume of peripheral blood stem cells place into the bioreactor will be at least 7X. While this at-least-seven-times-expansion is not necessary for this invention to work, this expansion is preferred for therapeutic purposes. For instance, the expanded cells may be only in amount of 2 times the number of peripheral blood stem cells placed in the rotating bioreactor, if desired. Preferably, expanded cells are in a range of about 4 times to about 25 times the number of peripheral blood stem cells placed in the bioreactor. In another preferred embodiment, the expanded cells number in an amount that is at least one cell more than the number that were placed in the culture chamber of the rotatable bioreactor. In this embodiment, the phenotypic expression of the cells after expansion is the preferred focus for repairing a body function or tissue.

The present invention is also directed to a composition comprising peripheral blood stem cells from a mammal, wherein said peripheral blood stem cells are expanded in a rotating TVEMF-bioreactor while suspending the cells therein to up or down regulate genes as effected by the cells environment, interactions, and three-dimensional geometry. A composition of the present invention may include a pharmaceutically acceptable carrier; plasma, peripheral blood, albumin, cell culture medium, growth factor, copper chelating agent, hormone, buffer or cryopreservative. “Pharmaceutically acceptable carrier” means an agent that will allow the introduction of the stem cells into a mammal, preferably a human. Such carrier may include substances mentioned herein, including in particular any substances that may be used for peripheral blood transfusion, for instance peripheral blood, plasma, albumin, preferably from the mammal to which the composition will be introduced. The term “introduction” of a composition to a mammal is meant to refer to “administration” of a composition to an animal. “Acceptable carrier” generally refers to any substance the peripheral blood stem cells of the present invention may survive in, i.e. that is not toxic to the cells, whether after TVEMF-expansion, prior to or after cryopreservation, prior to introduction (administration) into a mammal. Such carriers are well known in the art, and may include a wide variety of substances, including substances described for such a purpose throughout this application. For instance, plasma, peripheral blood, albumin, cell culture medium, buffer and cryopreservative are all acceptable carriers of this invention. The desired carrier may depend in part on the desired use.

Expanded peripheral blood stem cells have essentially the same, or maintain, the three-dimensional geometry and the cell-to-cell support and cell-to-cell geometry as the peripheral blood from which they originated. A preferred composition comprises expanded peripheral blood stem cells, preferably in a suspension of Dulbecco's medium or in a solution ready for cryopreservation. The composition is preferably free of toxic granular material, for example, dying cells and the toxic material or content of granulocytes and macrophages. The composition may be a cryopreserved composition comprising expanded peripheral blood stem cells by decreasing the temperature of the composition to a temperature of from −120° C. to −196° C. and maintaining the cryopreserved composition at that temperature range until needed for therapeutic or other use. As discussed below, preferably, as much toxic material as is possible is removed from the composition prior to cryopreservation.

Another embodiment of the present invention relates to a method of regenerating tissue and/or function with a composition of expanded peripheral blood stem cells, either having undergone cryopreservation or soon after expansion is complete. The cells may be introduced into a mammalian body, preferably human, for instance injected intravenously, directly into the tissue to be repaired, into the abdominal cavity, attaching to the peritoneum/peritoneal cavity, allowing the body's natural system to repair and regenerate the tissue. Preferably, the composition introduced into the mammalian body is free of toxic material and other materials that may cause an adverse reaction to the administered expanded peripheral blood stem cells.

An expanded peripheral blood stem cell composition of the present invention should be introduced into a mammal, preferably a human, in an amount sufficient to achieve repair of tissue and/or function, or to treat a desired disease or condition. Preferably, at least 20 ml of an expanded peripheral blood stem cell composition having 10⁷ to 10⁹ stem cells per ml is used for any treatment, preferably all at once, in particular where a traumatic injury has occurred and immediate tissue repair needed. This amount is particularly preferred in a 75-80 kg human. The amount of expanded peripheral blood stem cells in a composition being introduced into the source mammal is inherently related to the number of cells present in the source peripheral blood material. A preferred range of expanded peripheral blood stem cells introduced into a patient may be, for instance, about 10 ml to about 50 ml of an expanded peripheral blood stem cell composition having 10⁷ to 10⁹ stem cells per ml, or potentially even more. While it is understood that a high concentration of any substance, administered to a mammal, may be toxic or even lethal, it is unlikely that introducing all of a mammal's peripheral blood stem cells, for instance after expansion, will cause an overdose in expanded peripheral blood stem cells. Where peripheral blood from several donors is used, the number of peripheral blood stem cells introduced into a mammal may be higher. Therefore, it should be realized that the expanded cells may be introduced to the mammal from an allogeneic source or an autologous source. Also, the dosage of cells that may be introduced to the patient is not limited by the amount of peripheral blood provided from collection from one individual; multiple administrations, for instance once a day or twice a day, or once a week, or other administration time frames, may more easily be used. Also, where a tissue is to be treated, the type of tissue may warrant the use of as many expanded peripheral blood stem cells as are available.

The term “removing” refers to the physical removal of a mesenchymal stem cell product (discussed further below) from the culture chamber of the bioreactor. The mesenchymal stem cell product may be left in the solution they are in when removed from the culture chamber, the cells may be separated from culture medium and other solutions, the cells may be suspended in a different solution such as a buffer solution or a solution to cryopreserve the cells, and may otherwise be manipulated as desired. The mesenchymal stem cell product refers to particulate matter (preferably cells) removed from the bioreactor, and not to the solution the matter is in. The solution the cells are in may be discarded or used, for instance, chemicals in the solution may be recovered and used in other ways.

In operation, cells are placed into the culture chamber of a rotatable, preferably TVEMF, bioreactor. In one preferred embodiment, the culture chamber is rotated over a period of time, while at the same time a TVEMF is generated in the culture chamber by the TVEMF source. By “while at the same time,” it is intended that the initiation of the delivery of the TVEMF may be before, concurrent with, or after rotation of the culture chamber is initiated. Upon completion of the period of time, the expanded and/or differentiated cells are removed from the culture chamber. In a more complex rotatable TVEMF bioreactor, a culture medium enriched with culture medium requirements preferably including, but not limited to, buffer, nutrients, hormones, cytokines, and growth factors, which provides sustenance to the cells, can be periodically refreshed and removed.

In use, the present invention provides a stabilized culture environment into which cells may be introduced, suspended, assembled, grown, and maintained with improved retention of delicate three-dimensional structural integrity by simultaneously minimizing the fluid shear stress, providing three-dimensional freedom for cell and substrate spatial orientation, and increasing localization of cells in a particular spatial region for the duration of the expansion and/or differentiation. In use, the present invention also provides these three criteria (hereinafter referred to as “the three criteria above”), and at the same time, exposes the cells to a TVEMF. Of particular interest to the present invention is the dimension of the culture chamber, the sedimentation rate of the cells, the rotation rate, the external gravitational field, and the TVEMF.

The stabilized culture environment referred to in the operation of present invention is that condition in the culture medium, particularly the fluid velocity gradients, prior to introduction of cells, which will support a nearly uniform suspension of cells upon their introduction thereby creating a three-dimensional culture upon addition of the cells. In a preferred embodiment, the culture medium is initially stabilized into a near solid body horizontal rotation about an axis within the confines of a similarly rotating chamber wall of a rotatable TVEMF bioreactor. The chamber walls are set in motion relative to the culture medium so as to initially introduce essentially no fluid stress shear field therein. Cells are introduced to, and move through, the culture medium in the stabilized culture environment thus creating a three-dimensional culture. The cells move under the influence of gravity, centrifugal, and coriolus forces, and the presence of cells within the culture medium of the three-dimensional culture induces secondary effects to the culture medium. The significant motion of the culture medium with respect to the culture chamber, significant fluid shear stress, and other fluid motions, is due to the presence of these cells within the culture medium.

In most cases the cells with which the stabilized culture environment is primed sediment at a slow rate preferably under 0.1 centimeter per second. It is therefore possible, at this early stage of the three-dimensional culture, to select from a broad range of rotational rates (preferably of from about 2 to about 30 RPM) and chamber diameters (preferably of from about 0.5 to about 36 inches). Preferably, the slowest rotational rate is advantageous because it minimizes equipment wear and other logistics associated with handling of the three-dimensional culture.

Not to be bound by theory, rotation about a horizontal (or at least a substantially horizontal) axis with respect to the external gravity vector at an angular rate optimizes the orbital path of cells suspended within the three-dimensional culture. In operation, the cells expand and/or differentiate to form a mass of cell aggregates, three-dimensional tissues, and/or tissue-like structures, which increase in size as the three-dimensional culture progresses. The progress of the three-dimensional culture is preferably assessed by a visual, manual, or automatic determination of an increase in the diameter of the three-dimensional cell mass in the three-dimensional culture. An increase in the size of the cell aggregate, tissue, or tissue-like structure in the three-dimensional culture may require appropriate adjustment of the rotation speed in order to optimize the particular paths. The rotation of the culture chamber optimally controls collision frequencies, collision intensities, and localization of the cells in relation to other cells and also the limiting boundaries of the culture chamber of the rotatable TVEMF bioreactor. In order to control the rotation, if the cells are observed to excessively distort inwards on the downward side and outwards on the upwards side then the revolutions per minute (“RPM”) may preferably be increased. If the cells are observed to centrifugate excessively to the outer walls then the RPM may preferably be reduced. Not to be bound by theory, as the operating limits are reached, in terms of high cell sedimentation rates or high gravity strengths, the operator may be unable to satisfy both of these conditions and may be forced to accept degradation in performance as measured against the three criteria above.

The cell sedimentation rate and the external gravitations field place a lower limit on the fluid shear stress obtainable, even within the operating range of the present invention, due to gravitationally induced drift of the cells through the culture medium of the three-dimensional culture. Calculations and measurements place this minimum fluid shear stress very nearly to that resulting from the cells' terminal sedimentation velocity (through the culture medium) for the external gravity field strength. Centrifugal and coriolis induced motion [classical angular kinematics provide the following equation relating the Coriolis force to an object's mass (m), its velocity in a rotating frame (vᵣ) and the angular velocity of the rotating frame of reference (□): F_{Coriolis}=−2 m (w×vᵣ)] along with secondary effects due to cell and culture medium interactions, act to further degrade the fluid shear stress level as the cells expand and/or differentiate.

Not to be bound by theory, but as the external gravity field is reduced, much denser and larger three-dimensional structures can be obtained. In order to obtain the minimal fluid shear stress level it is preferable that the culture chamber be rotated at the same rate (or at least substantially the same rate) as the culture medium. Not to be bound by theory, but this minimizes the fluid velocity gradient induced upon the three-dimensional culture. It is advantageous to control the rate and size of tissue formation in order to maintain the cell size (and associated sedimentation rate) within a range for which the rate of expansion and/or differentiation is able to satisfy the three criteria above. However, preferably, the velocity gradient and resulting fluid shear stress may be intentionally introduced and controlled for specific research purposes such as studying the effects of shear stress on the three-dimensional cell aggregates. In addition, transient disruptions of the expansion and/or differentiation process are permitted and tolerated for, among other reasons, logistical purposes during initial system priming, sample acquisition, system maintenance, and culture termination.

Rotating cells about an axis perpendicular to (or at least substantially perpendicular to) gravity can produce a variety of sedimentation rates, all of which according to the present invention remain spatially localized in distinct regions for extended periods of time ranging from seconds (when sedimentation characteristics are large) to hours (when sedimentation differences are small). Not to be bound by theory, but this allows these cells sufficient time to interact as necessary to form multi-cellular structures and to associate with each other in a three-dimensional culture. Preferably, cells undergo expansion and/or differentiation for at least 4 days, more preferably from about 7 days to about 14 days, most preferably from about 7 days to about 10 days, even more preferably about 7 days. Preferably, TVEMF-expansion may continue in a rotatable TVEMF bioreactor to produce a concentration of cells per volume that is at least 7 times the original concentration of cells per volume that were placed in the rotatable TVEMF bioreactor.

Culture chamber dimensions also influence the path of cells in the three-dimensional culture of the present invention. A culture chamber diameter is preferably chosen which has the appropriate volume, preferably of from about 15 ml to about 2 L for non-perfused bioreactor, and about 100 ml to about 3 L for a perfused bioreactor, for the intended three-dimensional culture and which will allow a sufficient seeding density of cells. Not to be bound by theory, but the outward cells drift due to centrifugal force is exaggerated at higher culture chamber radii and for rapidly sedimenting cells. Thus, it is preferable to limit the maximum radius of the culture chamber as a function of the sedimentation properties of the tissues anticipated in the final three-dimensional culture stages (when the largest cell aggregates with high rates of sedimentation have formed).

The path of the cells in the three-dimensional culture has been analytically calculated incorporating the cell motion resulting from gravity, centrifugation, and coriolus effects. A computer simulation of these governing equations allows the operator to model the process and select parameters acceptable (or optimal) for the particular planned three-dimensional culture. FIG. 19 shows the typical shape of the cell orbit as observed from the external (non-rotating) reference frame. FIG. 20 is a graph of the radial deviation of a cell from the ideal circular streamline plotted as a function of RPM (for a typical cell sedimenting at 0.5 cm per second terminal velocity). This graph (FIG. 20) shows the decreasing amplitude of the sinusoidally varying radial cells deviation as induced by gravitational sedimentation. FIG. 20 also shows increasing radial cells deviation (per revolution) due to centrifugation as RPM is increased. These opposing constraints influence carefully choosing the optimal RPM to preferably minimize cell impact with, or accumulation at, the chamber walls. A family of curves is generated which is increasingly restrictive, in terms of workable RPM selections, as the external gravity field strength is increased or the cell sedimentation rate is increased. This family of curves, or preferably the computer model which solves these governing orbit equations, is preferably utilized to select the optimal RPM and chamber dimensions for the expansion and/or differentiation of cells of a given sedimentation rate in a given external gravity field strength. Not to be bound by theory, but as a typical three-dimensional culture is expanded and/or differentiated the tissues, cell aggregates, and tissue-like structures increase in size and sedimentation rate, and therefore, the rotation rate may preferably be adjusted to optimize the same.

In the three-dimensional culture, the cell orbit (FIG. 19) from the rotating reference frame of the culture medium is seen to move in a nearly circular path under the influence of the rotating gravity vector (FIG. 21). Not to be bound by theory, but the two pseudo forces, coriolis and centrifugal, result from the rotating (accelerated) reference frame and cause distortion of the otherwise nearly circular path. Higher gravity levels and higher cell sedimentation rates produce larger radius circular paths which correspond to larger trajectory deviations from the ideal circular orbit as seen in the non-rotating reference frame. In the rotating reference frame it is thought, not to be bound by theory, that cells of differing sedimentation rates will remain spatially localized near each other for long periods of time with greatly reduced net cumulative separation than if the gravity vector were not rotated; the cells are sedimenting, but in a small circle (as observed in the rotating reference frame). Thus, in operation the present invention provides cells of differing sedimentation properties with sufficient time to interact mechanically and through soluble chemical signals. In operation, the present invention provides for sedimentation rates of preferably from about 0 cm/second up to 10 cm/second.

Furthermore, in operation the culture chamber of the present invention has at least one aperture preferably for the input of fresh culture medium and a cell mixture and the removal of a volume of spent culture medium containing metabolic waste, but not limited thereto. Preferably, the exchange of culture medium can also be via a culture medium loop wherein fresh or recycled culture medium may be moved within the culture chamber preferably at a rate sufficient to support metabolic gas exchange, nutrient delivery, and metabolic waste product removal. This may slightly degrade the otherwise quiescent three-dimensional culture. It is preferable, therefore, to introduce a mechanism for the support of preferred components including, but not limited to, respiratory gas exchange, nutrient delivery, growth factor delivery to the culture medium of the three-dimensional culture, and also a mechanism for metabolic waste product removal in order to provide a long term three-dimensional culture able to support significant metabolic loads for periods of hours to months, and even longer.

In a preferred embodiment, the cells in the culture chamber are exposed to a TVEMF supplied by a TVEMF-source. The TVEMF-source may preferably comprise at least one loop, a coil, and/or a solenoid adapted to removably receive a rotatable bioreactor in an interior portion so that the TVEMF is applied to the cells in the culture chamber in an essentially uniform distribution. The TVEMF of the present invention may be exposed to the cell prior to, after, and/or during the expansion process.

In addition to the qualitatively unique cells that are produced by the operation of the present invention, not to be bound by theory, an increased efficiency with respect to utilization of the total culture chamber volume for cell and tissue culture may be obtained due to the uniform (or at least substantially uniform) homogeneous suspension achieved therein. Many mammalian cell types may be utilized in this method. Fundamental cell and tissue biology research as well as clinical applications requiring accurate in vitro models of in vivo cell behavior are applications for which the present invention and method of using the same provides an enhancement because, as indicated above and throughout this application, the expanded and/or differentiated cells and tissue of the present invention have essentially the same three-dimensional geometry and cell-to-cell support and cell-to-cell geometry as naturally-occurring, non-expanded cells and tissue.

The method of the present invention provides these three criteria above in a manner heretofore not obtained and optimizes a three-dimensional culture, and at the same time, facilitates and supports expansion such that a sufficient expansion (culture, increase in number, diameter in reference to tissue, and/or differentiation) is detected in a sufficient amount of time. The present invention is well adapted to carry out the objects and obtain the ends and advantages mentioned herein, as well as those inherent therein. Without departing from the scope of the invention, it is intended that all matter contained herein be interpreted as illustrative and not limiting.

### Operational Method

### Process for Preparing a Cellular Composition

The present invention is directed in part to a process for preparing a cellular composition. The process comprises the step of mixing a mesenchymal stem cell product with a biogel to prepare the cellular composition. The term “mixing” means placing a biogel and mesenchymal stem cell product into a container so that the biogel contacts at least one cell. Preferably, the biogel and the mesenchymal stem cell product are mixed thoroughly together, so that random samples of the cellular composition will have similar amounts of biogel and mesenchymal stem cell product. Preferably, the mesenchymal stem cell product, prior to mixing with the biogel, is free of culture medium, buffer, and other chemicals the product may have been in for instance during the expansion process or during storage of the product. Alternatively, the mesenchymal stem cell product includes a buffer solution or other solution wherein expanded cells are suspended. Conventional small and large scale systems available for mixing cellular material with biogel may be used to prepare the cellular composition.

Preferably, the biogel of the present invention is therapeutically acceptable. The term “therapeutically acceptable” means the biogel may be introduced into a mammalian body without causing a life-threatening adverse effect. Preferably, the biogel is approved for administration to a human by the United States Food and Drug Administration. A biogel of the present invention is preferably selected from at least one of the group comprising (or at least consisting of) hydrogel polymer, polymerized polyethylene glycol diacrylate, polylactic acid, polyglycolic acid, polymerized polyethylene glycol dimethylacrylate.

Also, preferably, the process further comprises the step of mixing a growth promoting agent with the biogel, the mesenchymal stem cell product, or a mixture of biogel and mesenchymal stem cell product. Such an agent may be mixed with the biogel prior to mixing with the mesenchymal stem cell product, and/or mixed with the mesenchymal stem cell product prior to mixing with the biogel, and/or mixed simultaneously with the mesenchymal stem cell product and the biogel. A growth promoting agent is preferably at least one of the group comprising (or at least consisting of) granulocyte colony-stimulating factor, vascular endothelial growth factor, transformation growth factor, bone morphogenetic protein and dexamethasone.

Also, preferably, the process further comprises the step of mixing a vascularization compound such as, for example, vascular endothelial growth factor (VEGF) with the biogel, the mesenchymal stem cell product, the growth promoting agent, or a mixture thereof.

Other additives may be included when preparing the cellular composition of the present invention. For instance, substances that cause the partial differentiation and/or complete differentiation of mesenchymal stem cells and/or other cells present in the cellular composition may be included in the cellular composition, as may substances that otherwise improve the consistency of the composition or improve the usefulness of the composition. Such additives may be added to the biogel prior to, concomitant with, and/or after mixing the biogel with the mesenchymal stem cell product. The additives may be added in a form and/or manner that allows the additives to immediately contact the cells of the mesenchymal stem cell product, or may be added in for instance a time-release form and/or manner, or other form and/or manner as desired.

The present invention is also directed to a cellular composition comprising a mesenchymal stem cell product and a biogel. Preferably, the biogel of the present invention is therapeutically acceptable. The term “therapeutically acceptable” means the biogel may be introduced into a mammalian body without causing a life-threatening adverse effect. Preferably, the biogel is approved for administration to a human by the United States Food and Drug Administration. A biogel of the present invention is preferably selected from at least one of the group comprising (or at least consisting of) hydrogel polymer, polymerized polyethylene glycol diacrylate, polylactic acid, polyglycolic acid, polymerized polyethylene glycol dimethylacrylate.

If the mesenchymal stem cell product is made from bone marrow stem cells, the source of the stem cells may be reflected in the product name, for instance by being called a bone marrow mesenchymal stem cell product. If the mesenchymal stem cell product is made from the bone marrow (or another acceptable source) of a mammal, and the resulting composition is to be administered to the same mammal, the mesenchymal stem cell product and resulting composition may be referred to as an autologous mesenchymal stem cell product. Alternatively, if the mesenchymal stem cell product is made from the bone marrow (or another acceptable source) of a mammal, and the resulting composition is to be administered to a different mammal, the mesenchymal stem cell product and resulting composition may be referred to as an allogeneic mesenchymal stem cell product.

Also, preferably, a cellular composition of the present invention further comprises a growth promoting agent. Such an agent may be mixed with the biogel prior to mixing with the mesenchymal stem cell product, and/or mixed with the mesenchymal stem cell product prior to mixing with the biogel, and/or mixed simultaneously with the mesenchymal stem cell product and the biogel. A growth promoting agent is preferably at least one of the group comprising (or at least consisting of) granulocyte colony-stimulating factor, vascular endothelial growth factor, transformation growth factor, bone morphogenetic protein and dexamethasone. Also, preferably, a cellular composition of the present invention further comprises a vascularization compound such as, for example, vascular endothelial growth factor (VEGF). Additives such as those discussed above may also be present in a cellular composition of the present invention.

### Operative Method

### Constructing Tissue, and Tissue Constructs

The present invention is directed in part to a method of constructing tissue, and tissue constructs. A cellular composition of the present invention may be used in many ways. The composition may be used to reconstruct tissue such as breast reconstruction after radical mastectomy. The composition may also be used for instance for breast augmentation, wrinkle removal, cosmetic applications, other tissue construction techniques. A growth factor and/or vascularization compound may be directly administered to the mammalian subject (preferably human) with the cellular composition. Such administration may occur at any time before, during and/or after the composition is administered.

The term “construct” and similar terms (such as construction, constructing) refers to replacing tissue or growing new tissue, and is meant to include tissue regeneration, reconstitution, replacement, growth, regrowth, augmentation and other terms that relate to the replacement or new growth of tissue. Construction may occur wholly in vivo (where a cellular composition of this invention is administered to the site where tissue growth is desired), wholly ex vivo (where a cellular composition is applied to an ex vivo structure and new tissue constructed on the structure, and then placed (with or without the structure) as desired on a mammal, or partly in vivo and partly ex vivo (for instance, tissue growth begins outside of the mammal's body and is completed within the mammal's body).

In a preferred embodiment, the cellular composition of the present invention may be used to construct tissue by differentiating mesenchymal stem cells and/or partially differentiated cells into differentiated cells to grow tissue. For instance, a cellular composition having biogel including a differentiating agent and VEGF may be used to form a three-dimensional structure of biocompatible tissue, such as adipose tissue. The present invention is intended to include a tissue construct (tissue grown from a cellular composition of this invention), a method of making the tissue construct, and a method for placing a cellular composition and/or tissue construct in or on a mammal.

The construction of tissue according to the present invention may provide tissue very similar to or the same as natural tissue in cellular and structural components, or may provide for tissue that is very different from naturally-occurring tissue. For instance, in the case of radical mastectomy where a breast has been removed from a human, the tissue that is constructed will preferably include mammary glands, mammary ducts, adipose tissue, blood vessels, lobules, and other histological characteristics of typical breast tissue. However, any tissue may be constructed to replace the lost breast by the cellular composition of the present invention. For instance, the constructed tissue may have more or fewer blood vessels than normal tissue, may have more adipose tissue, or may have connective tissue that is not normally found in breast tissue. Preferably in such a case, the constructed tissue is in the shape of a breast, and more preferably also has a similar volume as the remaining breast, or as desired by the patient. Similarly, breast implants for breast augmentation may be prepared, in vivo and/or ex vivo, by administering the present invention to a patient. Such implants will preferably have the benefit of comprising tissue constructed from the patient's own cells, and less likely to be for instance rejected by or toxic to the body.

A cellular composition of the present invention may be directly applied onto and/or into a site on a mammal, for instance a breast and/or other area where tissue growth is desired. The composition may also be used to grow tissue, for instance as in methods known in the art. The composition may be implanted in vivo in a mammal and the tissue grown entirely therein, or grown (in part or in full) ex vivo to produce a functional tissue or portion thereof, which may then be implanted in or otherwise administered to a mammal as desired.

While the mesenchymal stem cells are expanded (or thereafter), usually to a minimum level of seven times, they are preferably differentiated into the desired cells to be used for transplantation. In a preferred embodiment, the mesenchymal stem cells are cultured, in another preferred embodiment increased in number, and in another preferred embodiment cultured, differentiated and increased in number. After expansion, the mesenchymal stem cell product is then mixed with the aforestated biogel and growth factors and, if desired, VEGF and/or another vascularization agent to enhance growth and vascularization. If the biogel is a polyethylene glycol material, such as hydrogel polymer, it can then be formed into the desired shape (such as a breast to be reconstructed) and set in shape by conventional means (UV) before transplantation.

The example herein are for purposes of describing embodiments of the invention and are not intended to limit the invention more restrictive than that claimed.

### EXAMPLE

Typically, mesenchymal stem cells are cultured in conventional culture media, for example, modified Dulbecco's medium and supplemented with 5% human plasma and G-CSF. The components can be added to the culture chamber of the rotatable bioreactor or admixed prior to adding them to the culture chamber.

### Collection and Maintenance of Cells

Collected mesenchymal stem cells will be obtained from human donor bone marrow and/or blood by standard aspiration or blood drawing techniques known in the art. Blood includes peripheral and/or cord blood. Mesenchymal stem cells will be separated from other cells from the entire bone marrow and/or blood, and suspended in Iscove's modified Dulbecco's medium (IMDM) (GIBCO, Grand Island, N.Y.) supplemented with 5% human albumin (HA) or 20% human plasma, 100 ng/ml recombinant human G-CSF (Amgen Inc., Thousand Oaks, Calif.), and 100 ng/ml recombinant human stem cell factor (SCF) (Amgen). D-Penicillamine [D(−)-2-Amino-3-mercapto-3-methylbutanoic acid] (Sigma-Aldrich) a copper chelating agent, is dissolved in DMSO. 10 ppm of the D-Penicillamine is introduced into the cell mixture. The mesenchymal stem cell mix is to be placed into a culture chamber of a rotatable bioreactor as described herein. The rotatable bioreactor is to be rotated at a speed sufficient to insure the cells do not contact (or at least does not often contact) the walls of the culture chamber of the bioreactor. The cells should remain in suspension in the culture chamber.

After seven days of expansion including an increase in number and culture, the cells are to be washed with PBS and analyzed by conventional counting techniques, for example by using a Coulter counter. The cells are to be expanded until they have expanded at least seven times. The cells are then to be differentiated into the specific cells desired, mixed with a therapeutically acceptable biogel and preferably growth factors and vascularization compounds to form a cellular composition. The cellular composition may then be inserted/administered into the body for tissue construction, for instance by injection or by being set therein. (For instance, for wrinkle removal, injection is preferred; for breast implant, a volume of the cellular composition will be set inside the breast). The resulting mixture can be used for instance for breast regeneration, breast augmentation, wrinkle removal, cosmetic applications, and many other therapeutic tissue construction techniques.

When using a rotatable bioreactor with a perfused culture chamber, for instance as shown in FIGS. 4-7, a method of the current invention may be generalized as follows. An appropriate volume culture chamber and perfused rotatable bioreactor with a vane, such as a 500 ml culture chamber, will be chosen. The culture chamber will be first connected to the external culture media perfusion conduits including the gas exchange membranes, pump, and culture media sample ports and then sterilized, preferably with ethylene oxide gas, and washed with sterile phosphate buffered saline (PBS) then watered and aerated. The culture chamber will be completely filled with the appropriate culture media for the mesenchymal stem cells to be expanded. The controlled environment incubator that completely surrounds the rotatable bioreactor and in which it operates will be set for 5% CO₂, 21% oxygen, and 37° Celsius. The culture media perfusion loop will be set at a rate adequate to allow timely equilibration of the liquid culture media dissolved gases with the external controlled incubator environment.

Cell attachment substrates will be introduced either simultaneously or sequentially with cells into the culture chamber to give an appropriate density, such as 5 mg of beads per of ml of culture media, of attachment substrate for the anchorage dependent cells. The culture media will be then rotated about a horizontal axis. The rotation will begin at about 10 revolutions per minute, the slowest rate which produces a microcarrier bead orbital trajectory in which the beads do not accumulate appreciably at the vessel walls either by gravitational induced settling or by rotationally induced centrifugation. The culture chamber wall, mixing vane, and centerline spin filter will be also set at the same rotation (10 RPM) in order to provide essentially no relative motion of the culture media with respect to the culture chamber internal surfaces and therefore producing the minimal fluid velocity gradients and fluid shear stresses in the culture fluid dynamic environment.

It is recognized that in gravity the relative motion of the rotating fluid without any particles may be reduced to a level which can not be easily preserved; however, that when particles are placed into the rotating fluid that a measurable or visual change occurs and some relative motion occurs. However, the shear stress field which results is the minimum which has been found in a bioreactor for culturing mesenchymal stem cells, and therefore, permits the creation of three-dimensional expanded mesenchymal stem cells which has not heretofore been possible. This mixture of freely suspended attachment substrates within the rotating fluid culture media and rotating rotatable bioreactor components will be allowed to equilibrate for a short period of time, a time sufficient for transient flows to dampen out. The culture media perfusion rate will be set to zero during the cell loading and initial attachment (cells attaching to the microcarrier substrates) phase so as to retain the small cells within the vessel proper and not to draw them through the spin filter and external culture media perfusion loop where severe cell damage would occur. This initial loading and attachment phase may take 2 to 4 hours. The absence of perfusion induced mixing, nutrient delivery, waste product removal, and respiratory gas exchange during this period is well tolerated due to the small total amount of initial cellular metabolism and the brevity of this condition. The desired cells are gently injected into the rotating culture media over a short period of time, (2 minutes) so as to minimize cell damage while passing through the syringe and injection port. After injection of the cells is complete, the vessel outer wall is quickly (within 1 minute) returned to initial rotation (10 RPM) to match the angular rotational rate of the rest of the system and thereby returning the fluid shear stress field to the minimal level obtainable for these freely suspended cells and attachment substrates.

All free gas bubbles are then purged via a venting port to assure minimal disruption to the rotating fluid/substrate/cell mixture which would result from buoyant gas interfering with the near solid body rotating mixture. The cells and beads remain in nearly the same local region due to the similarity of their orbits and do not achieve a large net separation from each other. They are allowed to interact with three-dimensional freedom for spatial orientation within the rotating fluid and are not exposed to disruptive shear stresses, which would cause cell damage and limit the assembly of delicate three-dimensional structures.

After the initial loading and attachment phase (2 to 4 hours) the perfusion loop is set at a low flow speed (4.5 ml. per minute) that does not interfere with the initial three-dimensional assembly process. As the culture progresses the size and sedimentation rate of the assembled tissue and cells increases, the system rotational rates will be increased (increasing in increments of about 1 to 2 RPM from about 10 to about 20 RPM or more) in order to reduce the gravitationally induced orbital distortion (from the ideal circular streamlines) of the now increased (2 millimeter) diameter tissue pieces. The rotational rate will be increased until centrifugation is observed to cause accumulation of the cell or tissue pieces at the outer culture chamber walls that becomes detrimental to further three-dimensional growth of delicate structure. The rapid cell growth and increasing total metabolic demand will necessitate additional intermittent injections of nutrients. This injection is increased as necessary to maintain glucose and other nutrient levels. The culture may be allowed to progress beyond the point at which it is possible to select excellent particle orbits. At this point gravity has introduced constraints which will somewhat degrade performance in terms of fully three-dimensional low shear tissue culture.

Every 15 minutes during the total culture period the spin filter will be stopped and started at 15-second intervals for 1 minute in order to clear particles from the spin filter surface. This will prevent accumulation of substrates, cells, and debris on the filter. Samples of the growing tissue will be withdrawn as desired from a syringe attached to the sample port. The culture chamber outer wall may be temporarily stopped to allow practical handling of this syringe connected to the vessel. The expansion process will preferably be continued until the mesenchymal stem cells are expanded to at least seven times the number that were placed in the culture chamber before expansion. It is expected that the expanded cells will be genetic expression modified as a result of the suspension and expansion in the three-dimensional environment of the rotating rotatable bioreactor. It is expected that some genes are up regulated and that some are down regulated in response to the low shear stress and non-turbulent environment, for instance genes related to growth may be up regulated. The use of the expanded mesenchymal stem cells is discussed herein.

Having fully described this invention, we claim:

1. A composition comprising a mesenchymal stem cell product and a therapeutically acceptable biogel.

2. A composition as in claim 1 wherein the mesenchymal stem cell product comprises expanded mesenchymal stem cells that have been expanded in a rotatable bioreactor rotating about a substantially horizontal axis to suspend the cells in a three-dimensional environment therein essentially without shear stress and minimum turbulence, and to at least seven times the number that were placed in the bioreactor before expansion.

3. A composition as in claim 1 wherein the therapeutically acceptable biogel is at least one of the group consisting of hydrogel polymer, polymerized polyethylene glycol diacrylate, polylactic acid, polyglycolic acid, and polymerized polyethylene glycol dimethylacrylate.

4. A composition as in claim 1 wherein the mesenchymal stem cell product is a bone marrow mesenchymal stem cell product.

5. A composition as in claim 1 further comprising at least one growth-promoting agent selected from the group consisting of granulocyte colony-stimulating factor, vascular endothelial growth factor, transformation growth factor, bone morphogenetic protein and dexamethasone.

6. A method for preparing a mesenchymal stem cell product comprising the steps of:
collecting a mesenchymal stem cell from an individual;
placing the mesenchymal stem cell and culture medium in a culture chamber of a bioreactor;
expanding the mesenchymal stem cell in the culture chamber of the bioreactor by rotating the culture chamber about a substantially horizontal longitudinal axis to suspend the cells in a three-dimensional environment therein essentially without shear stress and minimal turbulence;
differentiating at least one mesenchymal stem cell into a differentiated cell in the culture chamber of the bioreactor; and
removing expanded cell from the culture chamber to prepare the mesenchymal stem cell product.

7. The mesenchymal stem cell product prepared by the method of claim 6.

8. A method of preparing a cellular composition comprising the step of mixing a therapeutically acceptable biogel with a mesenchymal stem cell product prepared by the method of claim 6 to prepare a cellular composition.

9. The cellular composition prepared by the method of claim 8.

10. Use of the cellular composition of claim 9 in the manufacture of a medicament for the construction of tissue.

11. The method as in claim 8 wherein the therapeutically acceptable biogel is at least one of the group consisting of hydrogel polymer, polymerized polyethylene glycol diacrylate, polylactic acid, polyglycolic acid, and polymerized polyethylene glycol dimethylacrylate.

12. A method as in claim 6 or 8 wherein the mesenchymal stem cell is a bone marrow stem cell.

13. A method as in claim 6 or 8 wherein the mesenchymal stem cells are autologous mesenchymal stem cells.

14. A method as in claim 6 wherein the expanding step further comprises expanding the number of mesenchymal stem cells to at least seven times greater than the number of mesenchymal stem cells that were placed in the culture chamber of the rotatable bioreactor, and wherein the method further comprises:
filling the culture chamber completely with a culture medium containing discrete suspension material and mesenchymal stem cells to initially establish a culturing environment;
controlling the rotation of said culture chamber such that the culture medium except during transient periods has the following simultaneous properties:
i. collocation of the discrete suspension material and mesenchymal stem cells,
ii. essentially no relative motion of said culture medium with respect to the boundaries of the chamber, and
iii. freedom for three dimensional spatial orientation of assemblies formed by the culturing of the cells; and
iv. determining the assembly trajectory, in response to said determining step: increasing the speed of rotation of the culture chamber if the assembly particles fall excessively inward and downward on the downward side of the rotation cycle and excessively outward and insufficiently upward on the upgoing side of the rotation cycle to prevent wall impact; or decreasing the speed of rotation of the culture chamber in response to excessive accumulation of the assemblies near the outer wall of the culture vessel so as not to restrict three dimensional assembly, and as the size of the assemblies increase beyond the capability to fully satisfy the above three properties by further increase of the speed of rotation, selecting a rotational rate to visually obtain minimal collision frequency and intensity.

15. A method as in claim 6 wherein the expanding step further comprises exposing the expanding cells to a time varying electromagnetic field.

16. A method as in claim 8 wherein the mixing step further comprises mixing at least one growth promoting agent selected from the group consisting of granulocyte colony-stimulating factor, vascular endothelial growth factor, transformation growth factor, bone morphogenetic protein and dexamethasone with the mesenchymal stem cell product and the therapeutically acceptable biogel.

17. A method of reconstructing human tissue comprising introducing a cellular composition into a mammal to reconstruct the tissue comprising the steps of
placing a mesenchymal stem cell and discrete suspension material in a culture chamber of a rotatable bioreactor;
expanding the mesenchymal stem cell in the culture chamber of the rotatable bioreactor by rotating the culture chamber about a substantially horizontal longitudinal axis to suspend the cells in a three-dimensional environment;
differentiating at least one mesenchymal stem cell into a differentiated cell in the culture chamber of the bioreactor;
removing cells from the culture chamber to prepare the mesenchymal stem cell product;
mixing the mesenchymal stem cell product with a therapeutically acceptable biogel to prepare a cellular composition; and
introducing the cellular composition into a human to reconstruct the tissue.

18. A method as in claim 17 wherein the therapeutically acceptable biogel is selected from the group consisting of hydrogel polymers, polymerized polyethylene glycol diacrylate, polylactic acid, polyglycolic acid, polymerized polyethylene glycol dimethylacrylate and mixtures thereof.

19. A method as in claim 17 wherein the cellular composition further comprises a growth promoting agent selected from the group consisting of granulocyte colony-stimulating factor, vascular endothelial growth factor, transformation growth factor, bone morphogenetic protein and dexamethasone.

20. A method as in claim 17 wherein the composition is reintroduced into the individual from which the mesenchymal stem cells were derived.
